(19) United States
(12) Patent Application Publication
(10) Pub. No.: US 2011/0027862 A1
(43) Pub. Date: Feb. 3, 2011

(54) SAMPLE STABILIZATION

(75) Inventors: Malcolm BATES, Cambridge (GB); Andrew Simon Goldsborough, Bethesda, MD (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/826,399

(22) Filed: Jun. 29, 2010

(30) Foreign Application Priority Data
Jun. 29, 2009 (GB) GB0911227.7
Apr. 8, 2010 (GB) GB1005923.6

(51) Int. Cl. C12N 7/00; C12N 5/071; C07H 21/02; C09K 3/00

(52) U.S. Cl. 435/235.1; 435/325; 536/23.1; 252/182.32

(57) ABSTRACT

This disclosure relates to reagents, compositions, methods for stabilising RNA in an RNA-containing sample by contacting the sample with guanidine and a metal ion to form a stabilised RNA-containing composition in which the metal ion is present at a concentration which is no more than 20 mM, and the metal ion is derived from a metal other than from a Group 1 or Group 2 metal.

### RELATED APPLICATIONS

This application claims priority to GB 0911227.7 filed Jun. 29, 2009, and GB 1005923.6 filed Apr. 8, 2010, which are hereby incorporated by reference in their entirety.

### FIELD OF THE INVENTION

The present invention relates to the stabilisation, purification and/or isolation of biomolecules, in particular RNA, including methods for stabilising RNA, compositions and kits for extracting RNA, and stabilised RNA-containing compositions.

### BACKGROUND OF THE INVENTION

The extraction of intact biomolecules from a biological sample is an essential part of many laboratory and clinical diagnostic procedures. The instability of biomolecules such as nucleic acids, proteins, carbohydrates and lipids is well known and their integrity depends on a large number of parameters such as the physiological condition of the sample prior to removal from its original environment, how quickly the sample was removed from its source, the rate of sample cooling, sample storage temperature, humidity and the biomolecule purification method. It is generally understood that the treatment of the biological sample before and during biomolecule purification can cause very important changes in the intactness and integrity of the sample analyte. For example it is well known that RNA in particular is an extremely labile molecule that becomes completely and irreversibly damaged within minutes if it is not handled correctly. For example in Protocols in Molecular Neurobiology (Humana Press, 1992), Vol 13, Chap. 13 pg 212, it states that “Note that RNA is very sensitive to heat in guanidine hydrochloride; perform all steps on ice or at −20° C. as indicated”. Although RNA is perhaps one of the more labile biomolecules, proteins including post-translational modifications, lipids, small molecules of less than 2000 daltons and DNA can also be subject to substantial degradative processes.

Although enzymes are responsible for the majority of degradative processes, the analyte, in particular RNA will always tend to spontaneously hydrolyse during storage or processing and this process is largely dependent on the storage conditions such as temperature, water content, pH, light and stability and molecular weight of the analyte molecule but may also be dependent on the quality of the reagents used.

One of the most common and simple approaches for successful storage is to reduce the temperature of the biological sample. Generally samples are stored at temperatures below room temperature (20-24° C.); protein solutions at 4° C. or −20° C., nucleic acids in freezers at −20° C. or −80° C., in dry-ice or in liquid nitrogen. Anti-microbial agents such as sodium azide may be added to control microbial growth.

It is well known that RNA is particularly sensitive to degradation by enzymes, spontaneous hydrolysis, divalent metal cation catalysed hydrolysis, alkali sensitivity and cross-linking in FFPE samples. Ribonucleases (“RNases”) are a large group of ubiquitous enzymes associated with many sources including microbes, human skin, dust and the content of cells and tissues. They are also readily released from intra-cellular vesicles during freeze-thawing. Certain tissues including the pancreas are known to be particularly rich in RNase A. RNase A is one of the most stable enzymes known, readily regaining its enzymatic activity following, for example, chaotropic salt denaturation making it extremely difficult to destroy. A high concentration of chaotrope such as guanidine (4-6M) is required to destroy RNase activity (Thompson. J. and Gillespie. D. Anal Biochem. (1987) 163:281-91).

There are several methods for inhibiting the activity of RNases such as using; (i) ribonuclease peptide inhibitors (“RNasin”) an expensive reagent only available in small amounts and specific for RNase A, B and C, (ii) reducing agents such as DTT and β-mercaptoethanol which disrupt disulphide bonds in the RNase enzyme, but the effect is limited and temporary as well as being toxic and volatile, (iii) proteases such as proteinase K to digest the RNases, but the transport of proteinases in kits and their generally slow action allows the analyte biomolecules to degrade, (iv) reducing the temperature to below the enzymes active temperature; commonly tissue and cellular samples are stored at −80° C. or in liquid nitrogen, (v) anti-RNase antibodies, (vi) precipitation of the cellular proteins including RNases, DNA and RNA using solvents such as acetone or kosmotropic salts such as ammonium sulphate, a commercialised preparation of ammonium sulphate is known as RNAlater™, (vii) detergents to stabilise nucleic acids in whole blood such as that found in the PAXgene™ DNA and RNA extraction kit (PreAnalytix GmbH) and (viii) chaotropic salts. A range of such chaotropic mixtures are set out in “RNA Isolation and Analysis”, Editor. Jones (1994) Chapter 2. A precipitation procedure is described in U.S. Pat. No. 5,817,798 for RNA isolation from a biological sample. The sample is lysed by mechanical or chemical means and then treated with a high concentration of a transition metal with a valency of at least +2. This treatment is intended to precipitate cellular contaminants and facilitate isolation of RNA.

The primary goal of sample preparation and analysis is to minimise changes to the analyte biomolecule introduced as a result of the pre-analytical procedure and sample purification so that the analytical result resembles as closely as possible the original in vivo complexity and diversity of the biomolecules so that assay sensitivity and specificity is optimised. Whilst there are various methods and products that are available to reduce pre-analytical variation, all suffer from various drawbacks making their use problematic or sub-optimal. Procedures that are effective at stabilising one class of biomolecules are often ineffective at stabilising others so that the technician is obliged to choose a specialised reagent and procedure for each biomolecule analyte. For example the PAXgene™ system (PreAnalytix) (U.S. Pat. Nos. 6,602,718 and 6,617,170) can be used for nucleic acids but not proteins, whilst cocktails of protease inhibitors help to protect proteins from degradation but not nucleic acids.

Generally, commercialised stabilisation reagents for nucleic acids such as RNAlater™, RNAprotect™ or the PAXgene™ stabiliser have the major drawback because the reagent must be removed from the sample prior to the sample lysis step. This is due to the incompatibility of the stabiliser with the lysis reagents, notably with the guanidine found in the majority of lysis reagents. Inconveniently it is not therefore possible to simply add the lysis solution directly to the sample in the stabilisation reagent. This problem increases the overall protocol time as extra steps are required but also increases the potential for contamination between samples when the same pair of forceps are used to remove the sample, which is commonly the standard method set out in the manufacturer's instructions. It is also very difficult or impossible to automate the removal of the stabilisation reagent from the biological sample necessitating manual intervention. Yields are also reduced because inevitably some of the analyte can not be recovered using forceps and will be discarded along with the stabilisation reagent and RNAlater™ has the unfortunate effect of causing the tissue to contract and harden making lysis significantly more difficult and therefore RNA yields reduced. Sample removal can also be a centrifugation step to pellet for example blood cells from the stabilisation mixture as set out in the RiboPure™ (Ambion) and PAXgene™ protocols, again necessitating several extra steps and increasing the opportunity for sample contamination and degradation.

It can also be extremely difficult to use RNAlater™ for stabilising viral nucleic acids in blood, serum and plasma, and it is not recommended by the manufacturer, again because it is necessary to remove the stabilising solution from the virus particles after centrifugation but prior to viral lysis, which can be technically demanding or impossible as the viral pellets are often not visible and can consequently easily be lost in the stabilisation reagent by aspiration.

Traditionally RNA degradation is avoided by keeping the contact time between the guanidine and the lysate containing the RNA to a minimum; sample lysis is generally immediately followed by separation of the RNA from the guanidine. Technically this has led to significant problems notably that sample lysis has to be immediately followed by RNA purification which is not always possible or desirable particularly with large numbers of samples, when the assay is a bDNA assay or when automation is involved. It is not always possible to purify RNA at the time or site where the sample is extracted, for example a biopsy from a hospital operating theatre or a blood sample from a doctors office. In these cases, the sample must be very carefully stored prior to RNA extraction, which might be carried out within as little as 30 minutes but would more commonly occur only after several hours or days. As a consequence, it has been necessary to develop separate sample storage conditions for each type of tissue and final use of the RNA. As already stated this generally involves using either a dedicated stabilisation solution such as RNAlater™ or PAXgene™ or simply immediately freezing the sample in liquid nitrogen. One of the disadvantages of RNAlater™ is that it only serves for the stabilisation of nucleic acids and not for sample lysis, therefore the nucleic acid sample has to be physically separated from the stabilisation reagent prior to lysis which is commonly carried out with guanidine. At least in the case of the PAXgene™ stabilisation reagent, incomplete removal of the stabiliser will negatively impact RNA yields during purification (PAXgene™ Blood RNA Kit Handbook, June 2005).

TABLE 1
Guanidine salts used in various commercialised RNA purification kits

| Guanidine Type | Manufacturer | Product |
|---|---|---|
| Guanidine HCl | Macherey Nagel | 740643 NucleoSpin ® Multi-8 |
| NH₂C(═NH)NH₂•HCl | | Virus RAV 1 buffer |
| CAS 50-01-1 | Invitrogen/Applied Biosystems | 4342792C TEMPUS ™ Blood |
| EINECS 200-002-3 | | RNA Tube |
| | | 4378916 Wash Tempus |
| | | solution |
| | Roche Applied Science | 12033674001 High Pure RNA |
| | | Tissue Kit |
| | | 11828665001 High Pure RNA |
| | | Isolation Kit |
| Guanidine | QIAGEN GmbH | RNeasy ™ |
| thiocyanate | | RNeasy PIus ™ |
| NH₂C(═NH)NH₂ | | AllPrep ™ |
| HSCN | | RNeasy Fibrous Tissue ™ |
| CAS 593-84-0 | | RNeasy Lipid ™ |
| EINECS 209-812-1 | | RNeasy Bacteria ™ |
| | | RNeasy Plant ™ |
| | Invitrogen Corp. (now Life | PureLink ™ |
| | Technologies) | MagMAX-96 Total RNA |
| | | Isolation |
| | | RecoverAll(TM) Total Nucleic |
| | | Acid Isolation Kit for FFPE |
| | Roche Applied Science | Cat. No. 03 330 591 001 |
| | | MagNA Pure LC RNA Isolation |
| | | Kit III (Tissue) |
| | | MagNA Pure Compact RNA |
| | | Isolation Kit (Tissue) |
| | | Cat. No. 03 172 627 001 |
| | | MagNA Pure LC mRNA |
| | | Isolation Kit II (Tissue) |
| | | Cat. No. 04 823 125 001 High |
| | | Pure FFPE RNA Micro Kit |
| | | Amplicor HCV (Roche) |
| | | Amplicor HIV (Roche) |
| | | Amplicor HBV (Roche) |
| | | Amplicor Chlamidia (Roche) |
| | Promega Corp | Z3051 SV RNA Lysis Buffer |
| | | Z305 MagneSil ® Total RNA |
| | | Mini-isolation system RNA |
| | | Lysis Buffer |
| | | Z305 PureYield ™ RNA Lysis |
| | | Buffer |
| | | Z305G Maxwell 16 Total RNA |
| | | Lysis Buffer |
| | Macherey Nagel | 740955.20 NucleoSpin ® RNA |
| | | II RA 1 buffer |
| | | 740962.20 NucleoSpin ® RNA |
| | | L RA 1 buffer |
| | | 740709.2 NucleoSpin ® 96 |
| | | RNA RA 1 buffer |
| | | 740956.10 NucleoSpin ® Virus |
| | | RAV 1 buffer |
| | Ambion | AM1912 RNAqueous ® Kit |
| | | AM1910 ToTALLY RNA ™ Kit |
| | Sigma-Aldrich | RTN10 GenElute ™ |
| | | Mammalian Total RNA |
| | | Purification |
| | | BTR1-1KT GenElute ™ |
| | | Bacterial Total RNA |
| | | Purification Kit |
| | GE Healthcare | Illustra ™ |
| | PreAnalytiX | PAXgene ™ Blood RNA Kit |
| | | Buffer BR2 and BR3 |

Tissue disruption refers to the process of breaking a large tissue sample up into particles that are small enough to be consequently lysed by the addition of a chaotrope solution. The disruption breaks the sample up into pieces that allow efficient release of the analyte.

Methods of disruption of the tissue or cells in the lysis buffer are very variable are generally optimised for the particular tissue. Frequently the sample is frozen in liquid nitrogen and then ground whilst still frozen in a mortar and pestle before the lysis solution is added to solubilise the powder. Alternatively the tissue is directly solubilised in the lysis buffer using a Polytron® (Brinkmann Polytron) or tissue homeginiser, a bead mill breaker (TissueRuptor, QIAGEN), PreCellys® (Bertin Technologies), a Dounce homegeniser, a French press extruder, vortexing, sonication, or a combination of methods such as a Polytron followed by reducing the viscosity of the lysate by repeatedly passing it through a needle and syringe. Yeast and bacteria are generally difficult to lyse due to a robust cell wall and these samples may need special treatment with enzymes such as zymolase and lysozyme that are capable of digesting the cell wall.

Mechanical methods, whilst they are efficient at disrupting tissues, typically lead to heating of the sample due to friction and other processes which can be catastrophic for the quality of the RNA if a guanidine based buffer is used. The amount of sample heating during disruption depends in part on the intensity and time required for mechanical grinding, for example tissues that are rich in connective tissue such as skin, muscle or plant tissues rich in lignin and cellulose generally require more intense lysis than softer tissues such as liver, brain or plant flowers. Unless care is taken to cool the sample during disruption, the RNA analyte will inevitably degrade. The amount of degradation depends on the overall time, the intensity of mechanical disruption, whether the instrument used for disruption is cooled, the temperature of the laboratory and the amount, type and volume of the biological sample in the lysis solution.

Compounding the problem of RNA degradation in lysed tissue samples is that in order to improve nucleic acid yields several RNA purification procedures and protocols such as the PAXgene™ Blood RNA kit (PreAnalytiX GmbH), SV Total RNA™ (Promega Corp) and the Amplicor HCV RNA purification kits require the sample to be heated in a guanidine containing lysis buffer. Whilst the heating step will improve the yield it inevitably leads to lower quality RNA. Lysed samples should consequently never be left longer in the lysis buffer than necessary due to RNA degradation.

Storage of the sample is defined as any period of time that exceeds the stated manufacturer's instructions or guidelines (provided with a commercialised extraction kit) for the sample lysis step. The sample lysis step is itself defined as the period from when the biological sample is first contacted by the lysis solution, commonly a guanidine solution, until the bulk of the lysate has been removed from the analyte. As one example using the QIAGEN RNeasy™ Mini kit (Catalogue No. 74104), storage refers to the period between when the sample is contacted with buffer RLT until the lysate is centrifuged through the RNeasy spin-column. If no specific time is specified in the manufacturer's instructions, then storage of the lysate is generally 30 minutes or longer and can be up to 60 minutes, 2 hours, 8 hours, one day, one week and may be as long as several months or years.

Unfortunately it is not all always possible to avoid incubating the mixture of guanidine and lysate, for example some commercialised kit protocols for the extraction of analyte from tissues and in particular viruses require a guanidine heating step in order to improve RNA yield. For example the Maxwell® 16 Total RNA Purification Kit, (Cat No. TB351) Promega Corp, requires a 3 minute incubation at 70° C. whilst the NucleoSpin Virus (Clontech Cat No. 740956.10) and Nucleospin RNA Virus F (Cat No. 740958), Macherey Nagel GmbH both require a 5 min incubation with RAV 1 guanidine lysis buffer at 70° C. to complete viral lysis. In addition, the latter two kits also require the addition of carrier RNA to the guanidine lysis buffer prior to heating so that both the analyte RNA and the carrier RNA will be degraded during this treatment. Furthermore carrier RNA has a limited shelf life in the RAV 1 Lysis buffer requiring that it is added shortly before its use. Additionally, the HCV Cobas Amplicor protocol also requires a heating step to lyse the virions and this lysis solution contains the RNA internal control which will be partly degraded during the heating step in contact with the guanidine.

As a result of its unparalleled chaotropic properties, guanidine has been used for several decades for the extraction of nucleic acids (see Sambrook et al., (1989) Molecular Cloning: A Laboratory Manual (2nd Ed.) Cold Spring Harbor University Press, NY) but paradoxically it is rarely, if ever used for storage of biomolecules at ambient temperatures because of its extremely poor conservation properties unless frozen at −80° C. This has greatly limited its application for use during storage, transport and archiving of biological specimens making it necessary to develop alternative stabilisation mixtures. The use of guanidine has therefore been generally limited to the lysis and denaturation of a biological sample followed by the immediate extraction of the analyte biomolecule away from the guanidine solution. Whilst guanidine is, with good reason, used to inactivate catabolic enzymes such as RNases, the major drawback of its use is that the analyte consequently has to be removed rapidly from the guanidine before RNA degradation begins.

The present invention aims to overcome these disadvantages. This disclosure provides, in part, methods for isolating RNA from various types of samples such that the integrity of the RNA is preserved during cell and/or tissue sample processing. As discussed below, may be accomplished using a reagent containing a chaotrope and a metal ion. Further advantages and embodiments are described below.

### BRIEF DESCRIPTION OF THE DRAWINGS

FIG. 1. 2% EtBr Agarose gel analysis of samples 1-4 demonstrating that the addition of 8 mM CuCl₂ notably improves the integrity of the RNA sample during storage and increases its yield.

### SUMMARY OF THE INVENTION

This disclosure relates, in part, to reagents, compositions, and/or methods for stabilising RNA in a biological sample (e.g., an RNA-containing sample) by contacting the sample with a chaotropic agent (e.g., guanidine and/or arginine) and one or more (e.g., one, two, three, four, etc.) metal ion(s) to form a stabilised RNA-containing composition. In some aspects, the one or more metal ion is present at a concentration which is no more than 20 mM (e.g., from about 1 mM to about 20 mM, from about 5 mM to about 20 mM, from about 5 mM to about 15 mM, etc.) and/or the one or more metal ion is derived from a metal other than from a Group 1 or Group 2 metal. In some aspects, a composition for extracting RNA from a biological sample is provided in which the composition comprises a chatropic agent (e.g., guanidine and/or arginine) and metal ions that may be mixed with the sample to provide a metal ion concentration of, for example, no more than 20 mM, less than 10 mM, at least 2.5 mM whereby the RNA is stabilised against degradation wherein the metal ion is derived from a metal other than from a Group 1 or Group 2 metal. The metal ion may be, for example, one or more of an ion of copper, zinc, iron, zirconium, erbium, indium, terbium, silver, gold, aluminium, tin, bismuth, lead or vanadium. The metal ion may be introduced into a composition (e.g., a chaotropic composition and/or the biological sample) as a metal salt (e.g., CuCl₂, Cu(CO₂CH₃)₂, CuCl, AuCl, FeCl₃, ZrCl₄, TbCl₃, (CF₃SO₃)₃In). In certain aspects the reagents, compositions, and/or methods provide to the biological sample (e.g., a stabilised RNA sample) a metal ion concentration of less than 10 mM, at least 2.5 mM, at least 2M and no more than 8M. In some aspects, the RNA is viral RNA, mRNA or miRNA. Certain methods described herein include at least one step of lysing the biological sample (e.g., such as an RNA-containing sample) in the presence of a chaotrope (e.g., guanidine and/or arginine) and at least one metal ion. The composition may be capable of both lysing the biological sample (e.g., a cell or tissue) and stabilising the RNA-containing sample. Thus, in certain aspects, a combination of a chaotropic agent (e.g., guanidine and/or arginine) and a source of metal ions for stabilising RNA during extraction of the RNA from a biological sample and methods for using the same are provided. In certain aspects, a combination of guanidine and a source of metal ions for stabilising RNA during extraction of the RNA from a biological sample is provided such that the chaotropic agent (e.g., guanidine and/or arginine) and metal ion are contacted with the biological sample to form a stabilised RNA-containing composition. Thus, a stabilised RNA-containing composition, which comprises RNA, guanidine and a metal ion, wherein the metal ion is present at a concentration which is no more than 20 mM and the metal ion is derived from a metal other than from a Group 1 or Group 2 metal is provided.

In certain aspects, kits comprising such reagents and compositions may be provided. In addition to the reagents and compositions described herein, the kits may further include instructions for lysis of the biological sample and/or preparing a stablised RNA-containing composition. The kits may provide a pre-mixed solution comprising a chaotropic agent (e.g., guanidine and/or arginine) for lysis of the biological sample and/or at least one metal ions for stabilising an RNA-containing composition. The kit may also provide a chaotropic agent (e.g., guanidine and/or arginine) as a solution for lysis of the biological sample and a source of metal ions as a separate concentrated solution for dilution in the solution of chaotropic agent (e.g., guanidine and/or arginine). The pre-mixed solution may be provided in a pre-filled clinical sample tube that is sealed at sub-atmospheric pressure for receiving blood directly from a patient. A kit of the invention may comprise, for example, a solid phase binding surface for binding the RNA. Kits may also include a solid phase binding surface (e.g., comprising silica). In some aspects, stabilised RNA-containing composition is used in a bDNA assay, and/or is contacted with a solid phase binding surface (e.g., comprising silica) for binding the RNA. Thus, the kit may also provide the stabilised RNA-containing composition to be used for a bDNA assay.

In some aspects, the biological sample (e.g., RNA-containing sample) is derived from blood, serum, plasma, mammalian tissue (e.g., liver, spleen, brain, muscle, heart, oesophagus, testis, ovaries, thymus, kidneys, skin, intestine, pancreas, adrenal glands, lungs, bone marrow, or a cancer sample, tumour, biopsy), a plant tissue (e.g., leaves, flowers, pollen, seeds, stems and roots of rice, maize, sorghum, palm, vines, tomato, wheat, barley, tobacco, sugar cane and Arabidopsis), bacteria (e.g., E. coli, Staphylococcus, Streptococcus, Mycobacterium, Pseudomonas, and bacteria that cause Shigella, Diptheria, Tetanus, Syphilis, Chlamydia, Legionella, Listeria and leprosy), and/or a virus (e.g., Norwalk, Rotavirus, Poliovirus, Ebola virus, Marburg virus, Lassa virus, HIV, HCV, Hantavirus, Rabies, Influenza, Yellow fever virus, Corona Virus, SARS, West Nile virus, Hepatitis A, C(HCV) and E virus, Dengue fever virus, toga, Rhabdo, Picorna, Myxo, retro, bunya, corona and reoviruses). In certain aspects, the biological sample (e.g., RNA-containing sample) may comprise muscle, heart, skin, and/or fixed tissue, and wherein the step of lysing is conducted at a temperature of about 18° C. to about 26° C. for at least about 5 minutes. The lysing step may, for example, be carried out in automated or semi-automated mechanical lysing apparatus.

In certain aspects, a combination of guanidine and a source of metal ions for stabilising RNA during extraction of the RNA from a biological sample is provided such that the chaotropic agent (e.g., guanidine and/or arginine) and metal ion are contacted with the biological sample to form a stabilised RNA-containing composition. Thus, a stabilised RNA-containing composition, which comprises RNA, guanidine and a metal ion, wherein the metal ion is present at a concentration which is no more than 20 mM and the metal ion is derived from a metal other than from a Group 1 or Group 2 metal is provided.

In certain aspects, a method for stabilising RNA in a sample using a chaotrope and a metal ion wherein the metal ion is present at a concentration of, for example, about 0.1 mM to about 20 mM, no more than about 8 M, at least about 2 M is provided. The method comprises contacting a sample with the chaotrope and the metal ion as described above. In some aspects, the chaotrope is guanidine (e.g., guanidine hydrochloride or thiocyanate guanidine) and/or arginine and is present at, for example, no more than about 8 M, at least about 2 M, or from at least about 2 M to no more than about 8 M. The metal ion may be derived from a metal other than from a Group 1 or Group 2 metal such as, for example, copper or iron (e.g., Cu²⁺ or Fe³⁺). The composition may be used, for example, to extract RNA from a biological sample. As such, a stabilised RNA-containing composition comprising RNA, guanidine, and a metal ion is provided. In certain aspects, the composition comprises a metal ion concentration of less than 10 mM (e.g., about 5 mM, about 2.5 mM) and the chaotrope (e.g., guanidine and/or arginine) concentration is at least 2M and no more than 8M in the RNA-containing sample. Kits comprising such reagents and compositions may also be provided. Methods for maintaining the integrity of RNA within a biological sample by adding to the sample a chaotrope (e.g., guanidine and/or arginine) and at least type of one metal ion as described herein are provided. The method may improve the integrity of the RNA by at least about 25% after about one day of storage at 37° C., and/or about 100% or about 500% after about eight days of storage at 37° C., as compared to the integrity of RNA in a sample that does not contain the metal ion. In some aspects, the integrity of the RNA may be measured using Q-RT-PCR.

Other aspects of the reagents, compositions, and methods provided herein are described below. Variations of such reagents, compositions, and methods are also contemplated as will be apparent to one of skill in the art.

### DETAILED DESCRIPTION OF THE INVENTION

The reagents, compositions and methods described herein provide for improved biomolecule processing. The biomolecule (e.g., RNA) may be contained within a sample, such as a biological sample, which is processed to modify the environment of the biomolecule (e.g., to isolate the biomolecule). The biomolecule is typically at risk of being modified (e.g., degraded) or recovered at low yield for a variety of reasons during processing, and the reagents, compositions and methods described herein typically serve to reduce that risk. Exemplary conditions of processing that may be improved using the reagents, compositions and methods described herein include, for example, analysis, archiving, extraction, handling, isolation, preservation, purification, storage, and/or transport of the biomolecule. The reagents, compositions and methods described herein may be used to stabilize, reduce the instability of, improve the stability of, maintain the integrity of, reduce degradation (including but not limited to substantial degradation), maintain the molecular weight of, and/or protect (e.g., from the effects of amines) the biomolecule during processing. The biomolecule may be any molecule that, for example, contains a nucleoside or nucleotide. Exemplary biomolecules may include at least one deoxyribonucleotide, ribonucleotide, monomer, deoxyribonucleotide or ribonucleotide dimer, deoxyribonucleotide or ribonucleotide oligomer, deoxyribonucleotide or ribonucleotide oligonucleotide, deoxyribonucleic acid (DNA), ribonucleic acid (RNA), oligonucleotide, genomic DNA, mitochondrial DNA, plasmid DNA, viral DNA, ribonucleic acid (RNA) (e.g., miRNA), piRNA, siRNA, tRNA, viriods, hnRNA, mRNA, rRNA (e.g., 5S, 5.8S, 16S, 18S, 23S and 28S rRNA species)), viral RNA (e.g., derived from for example HCV, West Nile Disease Virus, Foot and Mouth Disease Virus, Influenza, SARS, or HIV RNA), single-stranded RNA (ssRNA), double-stranded RNA (dsRNA), hybridised DNA, hybridised RNA, hybridized RNA and DNA (dsRNA:DNA), a mixture of single and double stranded nucleic acid sequences, ribozyme, aptamer, the product of a synthetic organic process (e.g., an oligo-synthesizer), a chimera of RNA and DNA, the product of an enzymatic reaction (e.g., in vitro RNA transcription, amplified RNA (aRNA) a PCR amplification, rolling circle amplification (RCA) or ligase chain reaction (LCR)), an internal control standard, and/or control nucleic acid (e.g., DNA, RNA). In one embodiment, the reagents and/or compositions may comprise a metal ion in a sample at a concentration that improves one or more conditions of processing the biomolecule. In some embodiments, this disclosure provides a composition comprising a metal ion and a chaotrope as a mixture that improves at least one condition of processing the biomolecule. Other advantages and embodiments of the reagents, compositions and methods for improved biomolecule processing may be derived from the description provided herein.

In one aspect, the reagents, compositions and methods comprise and/or involve the use of a metal ion. The metal ion, metal or metal salt may have one or more of the following characteristics: soluble (e.g., in water), stable in guanidine, does not precipitate with β-mercaptoethanol, non-toxic, relatively inexpensive, reduces RNA degradation in many or even all types of biological samples regardless of their source, transparent or lightly coloured in guanidine, does not negatively affect RNA binding to silica surfaces, reduces or does not affect contaminant binding, does not degrade RNA by catalysis or depurination, trace amounts do not inhibit molecular assays and enzymes such as reverse transcriptase, and allows the parallel purification of DNA and/or proteins if desired.

In order to determine the optimum selection and concentration of metal ion or salt, empirical tests such as those set out herein may be required with a variety of sample types such as blood, tissue and cells. The metal ion may be derived from a metal other than from a Group 1 or Group 2 metal of the Periodic Table, or Group 1 or Group 2 elements (“s-block elements”). Group 1 and Group 2 metals of the Periodic Table include, for example, Li, Na, K, Rb, Cs, Fr, Be, Mg, Ca, Sr, Ba and Ra. The metal ion may be derived from a transition metal or metalloid with a valence of +1, +2, or more. Such metals and/or metalloids may be derived from, for example, the Lanthanides such as Terbium and Erbium, Actinides, or Group 13 (Boron Group) such as Aluminium, Gallium and Indium, Group 14 (Carbon Group) including Tin and Lead, Group 15 (Nitrogen Group) such as Bismuth or even Group 16 such as Tellurium. A metal other than from a Group 1 or Group 2 metal of the Periodic Table from which the metal ion may be derived may include, for example, aluminum, bismuth, cadmium, chromium, copper, erbium, gold, indium, iron, lead, manganese, nickel, silver, terbium, tin, vanadiaum, zinc, zirconium. Thus, the metal may be an ion of, for example, aluminum, bismuth, cadmium, chromium, copper (e.g., CuCl, CuCl₂, CuCO₂CH₃, Cu(CO₂CH₃)₂), erbium (e.g., ErCl₃, Er₂(C₂O₄)₃, Er(CF₃SO₃)₃), gold, indium (e.g., InCl, InCl₂, InCl₃, In(CF₃SO₃)₃), iron (e.g., FeCl₂, FeCl₃), lead, manganese, nickel, silver (e.g., AgCl, AgCO₂CH₃), terbium, tin, vanadiaum, zinc (e.g., ZnSO₄, ZnCl₂, ZnI₂, Zn₃ (PO₄)₂, Zn(CO₂CH₃)₂), zirconium (e.g., ZrCl₄, ZrF₄), and/or mixtures thereof (e.g., CuCl₂/FeCl₂). It has surprisingly been found that by including ions of metals and/or metal salts of, for example, aluminum, bismuth, cadmium, chromium, copper, erbium, gold, indium, iron, lead, manganese, nickel, silver, terbium, tin, vanadiaum, zinc, zirconium, the compositions may be used to stabilize a biomolecule. It has surprisingly been found that by including metals and metal salts of Copper, Zinc, Iron, Zirconium, Erbium, Indium, Terbium, Silver, Gold, Aluminium, Tin, Bismuth, Lead, Cadmium and Vanadium, that a stabilising mixture can be obtained, but it was not found useful to add metals or the metal salts of Sodium, Potassium, Barium, Magnesium. In some embodiments, compositions may also comprise a chaotrope such as guanidinium. In one aspect, the present invention provides a method for stabilising a biomolecule in a biomolecule-containing sample by introducing into the sample a metal ion, and optionally a chaotrope such as guanidinium, to form a stabilised biomolecule-containing composition. Guanidinium is especially useful for the processing of RNA from biological samples such as cells, tissues, and/or extracts. In a first aspect, the present invention provides a method for stabilising RNA in an RNA-containing sample, which method comprises contacting the sample with guanidine and a metal ion to form a stabilised RNA-containing composition in which the metal ion is present at a concentration which is no more than 20 mM, wherein the metal ion is derived from a metal other than from a Group 1 or Group 2 metal of the Periodic Table. Other types of metals and metal ions that may be used, alone or in combination with a chaotrope such as guanidinium, may also be suitable as would be understood by one of skill in the art.

It will be evident to one skilled in the art that various metals and metal salts can be tested for their suitability in this invention by adding them at a range of final concentrations such as 10 μM, 50 μM, 100 μM, 500 μM, 1 mM, 5 mM, 10 mM, in a fixed amount of guanidine solution, conveniently 600 μl of Buffer RLT (QIAGEN) and using the metal/RLT mixture as a lysis solution for a tissue such as rat liver and as set out in Example 1. Comparisons of the stabilisation can be using a control solution of guanidine containing no metal or metal salt. Following incubation at, for example 37° C. for one day or more, the RNA is extracted according to a standard protocol such as RNeasy™ (QIAGEN) and its intactness analysed by gel electrophoresis or by using a Bioanalyser2100 (Agilent) or other suitable method such as Q-RT-PCR as described above. Yields can be determined by OD2601280 spectrometry. Once suitable metals and metal salts have been identified their optimum concentration can be determined by further such tests. The intactness of other biomolecules such as phosphoproteins can be determined using suitable anti-phospho antibodies and ELISA or mass spectrometry.

The amount of metal ion contacted with the sample should not be so much as to provide a concentration which causes precipitation of components of the sample. It is preferred that the metal ion concentration in the stabilised RNA-containing composition is less than 15 mM and more preferably less than 10 mM. The metal ion concentration may be at least 10 μM, generally at least 50 μM, advantageously at least 100 μM, preferably at least 500 μM, more preferably at least 1 mM particularly preferably at least 2.5 mM and most preferably at least 5 mM. A particularly useful metal ion concentration is approximately 8 mM. As described above, other concentrations of metal ion may also be suitable.

Metal ions can be derived from transition metals or metalloids with a valence of +1 (e.g. Cu, Ag, Hg, In) or +2 or more, or metals and metalloids from the Lanthanides such as Terbium and Erbium, Actinides, or Group 13 (Boron Group) such as Aluminium, Gallium and Indium, Group 14 (Carbon Group) including Tin and Lead, Group 15 (Nitrogen Group) such as Bismuth or even Group 16 such as Tellurium. The metal is not derived from either Group 1 or Group 2 elements (“s-block elements”).

A partial list of the salt that can be combined with the metal or metals, by way of example is: chloride, bromide, iodide, fluoride, sulphate, sulphide, sulphite, formate, acetate, propionate, trifluoromethanesulphate, carbonate, bicarbonate, bisulphate, bisulphite, chlorate, chlorite, chloroacetate, citrate, chromate, cyanate, bromate, oxide, phosphate, fluorophosphate, hexafluorophosphate, hydrogen difluoride, hydrogen sulfate, hydrosulfite, hypophosphite, iodide, iodate, hydroxide, metabisulphite, methanesulphonate, methoxide, nitrite, nitrate, phosphite, pyrophosphate, borate, tetraborate, thiosulfate, ascorbate, tartrate, perchlorate, acetylacetonate, lactate, oxalate, phosphide, gluconate and it will be understood by one skilled in the art that a very large range of metal salts are commercially available.

Preferably the metal or metal salt has one or more of the following deautures: (1) Is readily soluble in water or the chaotrope solution, (2) is not toxic or has low toxicity, (3) does not react with guanidine, as for example does bleach, (4) is stable in guanidine during storage and transportation, (5) either does not adversely effect RNA yield or increases RNA yield, (6) does not lead to the sample including both the analyte and the contaminants to precipitate out of solution or form large aggregates or complexes, (7) is transparent or lightly colored allowing its simple identification but is preferably not opaque allowing magnetic beads such as NucliSENS® easyMAG® (BioMerieux) to be observed, and/or (8) has no effect on, or otherwise enhances sensitive downstream applications such as Q-RT-PCR.

The invention can also be used in the absence of a biological sample such as tissue and cells by mixing a metal or metal salt such as CuCl₂ to pure guanidine hydrochloride or thiocyanate and then adding the RNA sample to be stabilised. In this way the RNA such as an internal control for a diagnostic test can be stored and transported at room temperature or on ice rather than frozen on dry ice.

Preferred metal salts include Cupric chloride (CuCl₂) (Sigma-Aldrich Cat. No. 203149), Copper (II) acetate (Cu(CO₂CH₃)₂) (Fluka Cat. 61145), Cuprous chloride (CuCl), Gold (I) chloride (AuCl), Ferric chloride (FeCl₃) (Sigma-Aldrich Cat. No. 451649), Zirconium tetrachloride (ZrCl₄) (Sigma-Aldrich Cat. No. 647640), Terbium (III) chloride (TbCl₃) and Indium(III) trifluoromethanesulfonate (CF₃SO₃)₃In) (Sigma-Aldrich Cat. No. 442151). Slightly less effective at protecting RNA in guanidine solutions are Zinc acetate, Zinc sulphate>Silver (I) chloride (Riedel-de Haën Cat. No. 10213), >Erbium trifluoromethanesulfonate (CF₃SO₃)₃Er) (Aldrich Cat. No. 425672) whilst Cupric oxide (CuO) (Sigma-Aldrich Cat. No. 203130) offered little or no advantage compared with guanidine alone.

Mixtures of metals and metal salts can also be used in varying amounts such as a 1:1 ratio of CuCl₂ and FeCl₂ or a 2:1 ratio of CuCl₂ and ZnSO₄ or a copper iron salt compound. The valence of the metal ion used can vary between +1, +2, +3 or +4 or be a mixture of two or more metal valences such as Cu (+1)/Cu (+2) as in a mixture of the salts CuCl and CuCl₂, or be a mixture of different metal ions with different valences such as Cu (+2)/Fe(3+) as in a mixture of CuCl₂ and FeCl₃. The metal ions and mixtures of metal ions can also be from different Periodic table blocks such as a d-block, p-block, f-block or a mixture for example of a d-block with a p-block element. Other ratios, such as 3:1, 4:1, 5:1, 6:1, 7:1, 8:1, 9:1, 10:1, 3:2 (e.g., from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 21:1 to about 10:1, from about 2:1 to about 8:1, from about 2:1 to about 6:1, from about 3:1 to about 10:1, from about 3:1 to about 8:1, from about 3:1 to about 6:1, from about 4:1 to about 10:1, from about 4:1 to about 8:1, from about 5:1 to about 10:1, etc.), and the like may also be suitable as would be known by one of skill in the art and as may be determined empirically using the method described in Example 19.

The results using metal salts are particularly surprising as it is well known that many metal ion solutions such as Lead, Magnesium and Manganese are very destructive to RNA and are indeed essential for not only ribozyme but also nuclease activity such as DNase I, mung bean nuclease and S1 nuclease. In Hecht (1996), pg 264, Bioorganic Chemistry Nucleic Acids, Oxford University Press it is stated “The fact that metal-dependent cleavage of RNA may occur even under mild conditions must always be kept in mind when carrying out experiments with RNA” and (pg 266) “Several metal ions—for example Pb²⁺, Eu³⁺, Zn²⁺, Fe²⁺ and Mn²⁺—can induce site-specific cleavage of different RNA's at neutral pH”. Other possible catalytic roles of metal ions in enzymatic and nonenzymatic cleavages of phosphodiester bonds has been reviewed (Yarus, M. (1993) FASEB J. 7, 31-39). Indeed chelators such as EDTA and EGTA are frequently added to RNA or RNA lysis solutions because they are assumed to reduce RNA degradation by removing metal ions. It was therefore especially unexpected to find that the addition of metal and metal salts to RNA led to an improvement whilst the addition of a chelator led to a reduction in RNA integrity compared with the control containing only guanidine (see Example 8 below). EDTA is also commonly added to blood draw tubes to stop unwanted blood coagulation by removing free Calcium ions, subsequent addition of guanidine lysis buffer would be expected to lead to an increase in RNA degradation in such tubes. Therefore a study of the state-of-the-art would clearly direct the user towards trying to remove metal ions from the RNA storage solution, it was therefore most unexpected that adding metals and metal ions to guanidine solutions containing RNA would improve RNA integrity. It is known that amines can function as metal ligands by donation of electron lone pairs and can complex and sequester metal ions. Certain of these metal ion complexes such as with Copper are well defined and can involve mixtures of both water and amine. We suggest that the reduction in RNA quality observed when adding EDTA to the sample lysate may be due to the removal of the metal ions that would otherwise bind and neutralise the degradative activity of the amines.

The metal ion is typically introduced or included into a sample at a concentration that serves to improve one or more conditions of bioprocessing. The concentration of the metal ion may be determined by any number of methods including, for example, colorimetric systems (e.g., phenathroline, L-cysteine functionalized gold nanoparticles, 4-(3,5-dibromo-2-pyridylazo)-N-ethyl-N-sulfopropylaniline), radiolabel detection (e.g., detection of one of the radioactive isotopes of copper), atomic absorption spectroscopy, and/or using the empirical tests as set out in Example 19. The most appropriate type of metal, mixture of metals, or concentration thereof may depend on one or more of the following variables: (i) sample phase (liquid, fatty, solid, hard), (ii) sample type (viral, bacterial, plant, animal), complexity (single-celled, multicellular, tissue), (iii) sample weight, surface area and/or density, (iv) fat, amine, and structural protein content (contractile muscle fibres, collagen and elastin), (v) lysate storage time and temperature, (vi) guanidine type and concentration, (vii) solid phase purification type (silica membrane or silica beads), and/or (viii) specific downstream application for the RNA (bDNA, Q-RT-PCR, Northern, in vitro translation). An approximation of the utility of any particular metal type, mixture of metals, and/or suitable concentration may be rapidly determined using the systems described herein (e.g., as set out in Example 19). Empirical tests may also be necessary with a variety of biological samples (e.g., a range of tissue types). There may also be a trade-off between the optimum metal type/concentration and the desired biomolecule (e.g., RNA) yield and quality thereof. For example, a metal ion at one concentration may provide optimum RNA stability but with a reduced yield compared with the same metal ion at a higher or lower concentration. It will be apparent to one skilled in the art that such empirical tests can be carried out for example, as set out in Example 19, taking into account such variables (e.g., i-viii above). The selection of the most appropriate metal and its concentration can be conveniently studied by assessing the RNA yield by OD260 nm spectrometry, its purity by OD260/280 nm ratio measurements, and its integrity by RNA electrophoresis using for example a Bioanalyser 2100 (Agilent, USA) or by Q-RT-PCR 3′/5′ ratio determination. Such methods are well known in the art. In the first instance, for any particular metal salt, a wide range of concentrations (e.g., about 0.1 mM to about 50 mM) should be tested to determine the approximate optimum concentration for the metal followed by more precise testing with a smaller range of metal concentration (e.g., about 5 mM to about 15 mM) before the optimum concentration is determined. It may also be necessary when using a mixture of metal ions (e.g., one, two or more, such as copper and iron) to determine the optimum concentration of each within the mixture (e.g., 2 mM copper, 4 mM iron).

The concentration of metal ion may refer to the amount in a sample at any point during processing of a biomolecule. For instance, concentration may refer to the amount of metal ion in a sample, starting material, stock solution (e.g., 2×, 5×, 10×, 25×, 50×, 100×), intermediate, starting concentration, or final concentration in the end product. Concentration may also refer to the amount of metal ion in a stock solution (e.g., source of metal ions) to be diluted to a useful intermediate or final concentration in a composition by combination with another component (e.g., guanidine) and/or sample (e.g., which may be a liquid or may be combined with a liquid). A suitable metal ion concentration is any amount of metal ion that provides for improved biomolecule processing. In some aspects, the processing of a biomolecule may be improved by introducing into a biomolecule-containing sample a metal ion at a final concentration of, for example, from about 0.1 mM to about 50 mM, such as about 0.01 mM, 0.05 mM, 0.1 mM, about 0.5 mM, about 1 mM, about 2 mM, about 3 mM, about 4 mM, about 5 mM, about 0.1 mM to 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM. Suitable ranges of metal ion concentrations may be, for example, about 0.01 mM to about 0.05 mM, about 0.05 mM to about 1 mM, about 0.1 mM to about 1 mM, about 0.1 mM to about 1 mM, about 0.1 mM to about 5 mM, about 0.1 mM to about 10 mM, about 0.1 mM to about 20 mM, about 1 mM to about 5 mM, about 2 mM to about 3 mM, about 2 mM to about 4 mM, about 2 mM to about 5 mM, about 2 mM to about 6 mM, about 2 mM to about 7 mM, about 2 mM to about 8 mM, about 2 mM to about 9 mM, about 2 mM to about 10 mM, about 2 mM to about 11 mM, about 2 mM to about 12 mM, about 3 mM to about 4 mM, about 3 mM to about 5 mM, about 3 mM to about 6 mM, about 3 mM to about 7 mM, about 3 mM to about 8 mM, about 3 mM to about 9 mM, about 3 mM to about 10 mM, about 3 mM to about 11 mM, about 3 mM to about 12 mM, about 4 mM to about 5 mM, about 4 mM to about 6 mM, about 4 mM to about 7 mM, about 4 mM to about 8 mM, about 4 mM to about 9 mM, about 4 mM to about 10 mM, about 4 mM to about 11 mM, about 4 mM to about 12 mM, about 5 mM to about 6 mM, about 5 mM to about 7 mM, about 5 mM to about 8 mM, about 5 mM to about 9 mM, about 5 mM to about 10 mM, about 5 mM to about 11 mM, about 5 mM to about 12 mM, about 6 mM to about 7 mM, about 6 mM to about 8 mM, about 6 mM to about 9 mM, about 6 mM to about 10 mM, about 6 mM to about 11 mM, about 6 mM to about 12 mM, about 7 mM to about 8 mM, about 7 mM to about 9 mM, about 7 mM to about 10 mM, about 7 mM to about 11 mM, about 7 mM to about 12 mM, about 8 mM to about 9 mM, about 8 mM to about 10 mM, about 8 mM to about 11 mM, about 8 mM to about 12 mM, about 9 mM to about 10 mM, about 9 mM to about 11 mM, about 9 mM to about 12 mM, about 10 mM to about 11 mM, about 10 mM to about 12 mM, about 11 mM to about 12 mM, about 12 mM to about 13 mM, about 12 mM to about 14 mM, about 12 mM to about 15 mM, about 12 mM to about 16 mM, about 12 mM to about 17 mM, about 12 mM to about 18 mM, about 12 mM to about 19 mM, about 12 mM to about 20 mM, about 13 mM to about 14 mM, about 13 mM to about 15 mM, about 13 mM to about 16 mM, about 13 mM to about 17 mM, about 13 mM to about 18 mM, about 13 mM to about 19 mM, about 13 mM to about 20 mM, about 14 mM to about 15 mM, about 14 mM to about 16 mM, about 14 mM to about 17 mM, about 14 mM to about 18 mM, about 14 mM to about 19 mM, about 14 mM to about 20 mM, about 15 mM to about 16 mM, about 15 mM to about 17 mM, about 15 mM to about 18 mM, about 15 mM to about 19 mM, about 15 mM to about 20 mM, about 16 mM to about 17 mM, about 16 mM to about 18 mM, about 16 mM to about 19 mM, about 16 mM to about 20 mM, about 17 mM to about 18 mM, about 17 mM to about 19 mM, about 17 mM to about 20 mM, about 18 mM to about 19 mM, about 18 mM to about 20 mM, about 19 mM to about 20 mM, more than about 2 mM to less than about 20 mM, about 21 mM to about 22 mM, about 21 mM to about 23 mM, about 21 mM to about 24 mM, about 21 mM to about 25 mM, about 21 mM to about 26 mM, about 22 mM to about 23 mM, about 22 mM to about 24 mM, about 22 mM to about 25 mM, about 22 mM to about 26 mM, about 22 mM to about 27 mM, about 23 mM to about 24 mM, about 23 mM to about 25 mM, about 23 mM to about 26 mM, about 23 mM to about 27 mM, about 23 mM to about 28 mM, about 24 mM to about 25 mM, about 24 mM to about 26 mM, about 24 mM to about 27 mM, about 24 mM to about 28 mM, about 24 mM to about 29 mM, about 25 mM to about 26 mM, about 25 mM to about 27 mM, about 25 mM to about 28 mM, about 25 mM to about 29 mM, or about 25 mM to about 30 mM. Additionally suitable ranges of metal ion concentrations may be, for example, from about 1 mM to about 100 mM, about 1 mM to about 75 mM, about 1 mM to about 50 mM, about 1 mM to about 40 mM, about 1 mM to about 30 mM, about 1 mM to about 20 mM, about 1 mM to about 15 mM, about 1 mM to about 12 mM, about 1 mM to about 10 mM, about 2 mM to about 50 mM, about 2 mM to about 35 mM, about 2 mM to about 25 mM, about 2 mM to about 20 mM, about 2 mM to about 15 mM, about 2 mM to about 10 mM, about 4 mM to about 10 mM, about 5 mM to about 10 mM, about 5 mM to about 50 mM, about 10 mM to about 40 mM, or about 10 mM to about 25 mM. In some aspects, the metal ion concentration may be at least about 0.01 mM, at least about 0.05 mM, at least about 0.1 mM, at least about 0.5 mM, at least about 1 mM, at least about 2.5 mM, or at least about 0.05 mM. In other aspects, the processing of a biomolecule may be improved by introducing into a biomolecule-containing sample a metal ion at a final concentration of, for example, less than about 20 mM; less than about 10 mM; about 8 mM; and 8.2 mM; and, optionally a chaotrope such as guanidinium or argninne, to improve biomolecule processing. In some aspects, the biomolecule is RNA isolated from a biological sample. In a further aspect, the present invention provides a composition for extracting RNA from a biological sample, which composition comprises guanidine and a source of metal ions for mixing with the sample to provide a metal ion concentration of no more than 20 mM whereby the RNA is stabilised against degradation, wherein the metal ion is derived from a metal other than from a Group 1 or Group 2 metal. In a further aspect, the present invention provides a composition for stabilising RNA within a biological sample, which composition comprises a chaotrope at a suitable concentration and metal ions at a concentration of less than about 20 mM; less than about 10 mM; about 8 mM; or 8.2 mM. In a further aspect, the present invention provides a composition for extracting RNA from a biological sample, which composition comprises guanidine and about 8 mM copper (e.g., Cu²⁺) or iron (e.g., Fe³⁺). In a further aspect, the present invention provides a composition for extracting RNA from a biological sample, which composition comprises guanidine and about 8.2 mM copper (e.g., Cu²⁺) or iron (e.g., Fe³⁺). Other concentrations of metal ions may also be suitable, alone or in combination with a chaotrope such as guanidinium, as would be understood by one of skill in the art. Any of these suitable concentrations of metal ion may be combined with any suitable concentration of chaotrope.

As mentioned above, the metal ion may also be used with another component, such as a chaotrope (e.g., arginine or guanidine). Chaotropes destroy the hydrogen-bonding network of water, allowing macromolecules greater structural freedom and encouraging protein denaturation. Chaotropes function by readily disrupting inter- and intra-molecular hydrogen bonding, hydrophobic interactions and Van der Waals interactions thereby destroying the enzymatic activity of nearly all known enzymes and in particular RNases. This disruptive activity is not limited to RNases; proteases and other catabolic enzymes will, in general, all be disrupted by a chaotrope. Salts that are effective at precipitating proteins such as ammonium sulphate are generally poor chaotropes according to the Hoffmeister Series (Baldwin, R. L. Biophys J (1996). 71:2056-63). Ions that tend to denature proteins are I⁻, ClO⁴⁻, SCN⁻, Li⁺, Mg²⁺, Ca²⁺, Ba²⁺ and the guanidinium ion. Guanidinium is a planar ion that may establish strong hydrogen-bonded ion pairs to protein carboxylates. Guanidinium also possesses rather hydrophobic surfaces that may interact with similar protein surfaces to enable protein denaturation (P. E. Mason, G. W. Neilson, J. E. Enderby, M. L. Saboungi, L E. Dempsey, A. D. MacKerell Jr and J. W. Brady. J. Am. Chem. Soc. (2004) 22:11462-70). Other than destroying enzyme activity, chaotropes serve another essential function for analyte analysis namely the lysis and homogenisation of tissues and cells, thereby rendering the analyte accessible for purification and extraction away from contaminating molecules. It should be noted that the type of ‘contamination’ depends on the analyte; proteins are the contaminant of most nucleic acid analytes whilst nucleic acids are a common contaminant of protein analytes. Lastly, chaotropes and in particular guanidine salts in combination with alcohol have the desirable feature of promoting binding of nucleic acids to silica surfaces (Boom, R. et al. J. Clin. Microbiol. (1990) 28:495-503). Guanidine remains by far the most commonly used chaotrope due to its extremely powerful chaotropic activity and partly for long standing historic reasons (see Sambrook et al., (1989) Molecular Cloning: A Laboratory Manual (2nd Ed.) Cold Spring Harbor University Press, NY) and in particular its traditional use for this purpose. As shown herein, a chaotrope may be combined with a metal ion to provide a composition that may be used to improve processing of biomolecules. Suitable chatropes include but are not limited to those described herein or elsewhere, as would be understood by one of skill in the art.

A chaotrope may be combined with a metal ion such that the final concentration of the chaotrope in a sample is suitable to improve processing of the biomolecule (e.g., to improve the stability, integrity, etc. of RNA). Suitable chaotropes include, for example, guanylguanidinium, carbamoylguanidinium, biguanide, guanidine HCl, (NH₂C(═NH)NH₂.HCl, CAS 50-01-1, EINECS 200-002-3), guanidine thiocyanate (NH₂C(═NH)NH₂.HSCN, CAS 593-84-0, EINECS 209-812-1); Table 1), arginine (e.g., 2.7M L-Arginine pH 7.0 (Sigma-Aldrich Cat. No. 11009)), guanylguanidinium, carbamoylguanidinium (Castellino, F. J. and Barker R. Biochem. (1968) 7:4135-8), the synthetic molecule biguanide (CAS 4761-93-7), or combinations thereof. Aqueous solutions of such chaotropes are typically used. Exemplary, suitable final concentrations of chaotrope may be, for example, from about 0.1 to about 10 M, such as 0.1 M, 0.5 M, 1 M, 1.5 M, 2 M, 2.5 M, 2.7 M, 3 M, 3.5 M, 4 M, 4.5 M, 5 M, 5.5 M, 6 M, 6.5 M, 7 M, 7.5 M, 8 M, 8.5 M, 9 M, 9.5 M, 10 M, 10.5 M, 11 M, 11.5 M, 12 M, 12.5 M, 13 M, 13.5 M, 14 M, 14.5 M, 15 M, 15.5 M and 16 M. Suitable chaotrope concentration ranges include, for example, from about 2 M to about 3 M, about 2 M to about 4 M, about 2 M to about 5 M, about 2 M to about 6 M, about 2 M to about 7 M, about 2 M to about 8 M, about 2 M to about 9 M, about 2 M to about 10 M, about 2 M to about 11 M, about 2 M to about 12 M, about 2 M to about 13 M, about 2 M to about 14 M, about 2 M to about 15 M, about 2 M to about 16 M, about 3 M to about 4 M, about 3 M to about 5 M, about 3 M to about 6 M, about 3 M to about 7 M, about 3 M to about 8 M, about 3 M to about 9 M, about 3 M to about 10 M, about 3 M to about 11 M, about 3 M to about 12 M, about 3 M to about 13 M, about 3 M to about 14 M, about 3 M to about 15 M, about 3 M to about 16 M, about 4 M to about 5 M, about 4 M to about 6 M, about 4 M to about 7 M, about 4 M to about 8 M, about 4 M to about 9 M, about 4 M to about 10 M, about 4 M to about 11 M, about 4 M to about 12 M, about 4 M to about 13 M, about 4 M to about 14 M, 4 M about to about 15 M, about 4 M to about 16 M, about 5 M to about 6 M, about 5 M to about 7 M, about 5 M to about 8 M, about 5 M to about 9 M, about 5 M to about 10 M, about 5 M to about 11 M, about 5 M to about 12 M, about 5 M to about 13 M, about 5 M to about 14 M, about 5 M to about 15 M, about 5 M to about 16 M, about 6 M to about 7 M, about 6 M to about 8 M, about 6 M to about 9 M, about 6 M to about 10 M, about 6 M to about 11 M, about 6 M to about 12 M, about 6 M to about 13 M, about 6 M to about 14 M, about 6 M to about 15 M, about 6 M to about 16 M, about 7 M to about 8 M, about 7 M to about 9 M, about 7 M to about 10 M, about 7 M to about 11 M, about 7 M to about 12 M, about 7 M to about 13 M, about 7 M to about 14 M, about 7 M to about 15 M, about 7 M to about 16 M, about 8 M to about 9 M, about 8 M to about 10 M, about 8 M to about 11 M, about 8 M to about 12 M, about 8 M to about 13 M, about 8 M to about 14 M, about 8 M to about 15 M, about 8 M to about 16 M, about 9 M to about 10 M, about 9 M to about 11 M, about 9 M to about 12 M, about 9 M to about 13 M, about 9 M to about 14 M, about 9 M to about 15 M, about 9 M to about 16 M, about 10 M to about 11 M, about 10 M to about 12 M, about 10 M to about 13 M, about 10 M to about 14 M, about 10 M to about 15 M, about 10 M to about 16 M, about 11 M to about 12 M, about 11 M to about 13 M, about 11 M to about 14 M, about 11 M to about 15 M, about 11 M to about 16 M, about 12 M to about 13 M, about 12 M to about 14 M, about 12 M to about 15 M, about 12 M to about 16 M, about 13 M to about 14 M, about 13 M to about 15 M, about 13 M to about 16 M, about 14 M to about 15 M, about 14 M to about 16 M, and about 15 M to about 16 M. A suitable final concentration of chaotrope may also be from 2M to 8M, greater than about 2M, about 2.7 M, not more than about 8M, or greater than about 2M and less than about 8M. Any of these suitable concentrations of chaotrope may be combined with any suitable concentration of metal ion salt. Other components (e.g., water, buffer, chelator(s)) may also be included in the reagents and compositions described herein. The chaotrope and metal ion may be introduced into the biological sample together or separately to produce suitable final concentrations therein. Other final concentrations of the chaotrope may also be suitable as would be understood by one of skill in the art.

Despite the long felt need to preserve RNA and other biomolecules in guanidine lysis solutions, it has not been possible, until now to do so unless the sample is frozen at −80° C. Guanidine is extensively used because it is not only efficient at inhibiting catabolic enzymes, lysing tissues, cells and viruses but is also highly effective at releasing nucleic acids bound in nucleoprotein complexes which must be disrupted prior to successful nucleic acid purification. Extracting biomolecules from whole tissues is generally more difficult than from cells due to the presence of structural proteins such as collagen and elastin which tend to form aggregates except in the presence of strong chaotropes and/or proteases. These aggregates can, for example block the purification device such as a silica spin column thereby reducing the yield and purity of the analyte nucleic acid. It is therefore important to remove such structural proteins either by protease treatment or to solubilise them such that they can easily pass through the silica spin column without blockage. The simplest manner to do this is to use a strong chaotrope, in particular guanidine. Guanidine is nearly always used as either its thiocyanate, (Chirgwin, J. M. et al. Biochem. (1979) 18:5294-9) or hydrochloride forms. Its ionic form is known as the guanidinium ion. It is commonly used in combination with a reducing agent such as 1% β-mercaptoethanol to aid protein denaturation. Another reagent with a strong denaturant characteristic has been found to be the amino acid arginine which behaves in a similar chaotropic manner to guanidine hydrochloride. It is preferred that the guanidine concentration in the stabilised RNA-containing composition is at least 2M. This allows stabilisation of the RNA and enables RNA to be bound by a solid phase such as silica, if required. The invention is not particularly limited to any one salt of guanidine such as guanidine isothiocyanate or guanidine hydrochloride, neither is it particularly limited to any one type of guanidinium ion such as guanidine, guanylguanidinium or carbamoylguanidinium (Castellino, F. J. and Barker R. Biochem. (1968) 7:4135-8). Other forms of the guanidinium ion include the side chain of the amino acid arginine and the synthetic molecule biguanide (CAS 4761-93-7). Other salts of guanidine may also be suitable as would be understood by one of skill in the art. The term guanidine, as used herein, may also refer to a salt of guanidine. As described above for chaotropes generally, many concentrations, or ranges of concentrations, of guanidine or a salt thereof, may be suitable for use depending on a particular process being utilized. It is preferred that the guanidine concentration does not exceed 8M. For example, guanidine may be used at a final concentration in the sample of at least about 2M and less than about 8M, or at least about 2M and less than about 8M. Arginine may be used at, for example, a final concentration of about 2.7 M. Any of these suitable concentrations of chaotrope may be combined with any suitable concentration of metal ion salt. Other final concentrations of chaotrope may also be suitable as would be understood by one of skill in the art.

Preferably the mixture of metal or metal salt/chaotrope (e.g. guanidine) is stable meaning that the stabilisation activity of the mixture does not significantly change during storage of one month or more at ambient temperature. It is also preferred that the addition of the metal or metal salt does not increase the toxicity of the chaotrope or render it too viscous to manipulate or change colour significantly during preparation or storage. If the mixture is stable for less than month at ambient temperature and its stabilisation properties particularly good then small amounts of the mixture can be prepared from a stock concentrate of the metal or metal salt and the chaotrope to provide enough for immediate use or within a several days. It may be necessary to redissolve the metal or metal salt back into solution after prolonged standing by mixing and warming the solution.

It is also preferred that the use of the metal/guanidine mixture does not significantly reduce RNA binding to solid phase capture surfaces such as silica beads, magnetic silica beads or membranes. Metal addition to the guanidine should be sufficient to provide RNA protection whilst not so much that either the RNA precipitates out of solution and is therefore lost, or is inhibited from binding to the solid phase. The metal should also not contaminate the solid phase binding surface and reduce sensitivity of the downstream application such as RT-PCR. In the application of bDNA assays, the amount of metal added should also not be so much that hybridisation to the bDNA probes be inhibited. Appropriate amounts of metal salt addition are most simply found by empirical means as set out above.

Proteinase K is active in guanidine HCl and thiocyanate solutions but requires an incubation step at 37-60° C. for several minutes to hours in order to be able to digest protein structures and therefore release RNA and DNA from the sample. It is highly beneficial to add a metal salt to the guanidine/Proteinase K solution in order to reduce the RNA degradation that would otherwise occur at these extreme reaction temperatures required this incubation step allowing higher quality RNA and better yields of RNA and DNA. Similarly for RNA extraction from FFPE samples, the RNA can be guarded in a more intact state when heating is necessary to reverse cross links during purification.

Many different methods may be used with the reagents and compositions described herein. For example, to isolate RNA from a tissue sample, a metal ion/guanidinium composition may be prepared by adding to a guanidine-containing buffer (e.g., Buffer RLT (QIAGEN)) may be added a solution of a metal ion (e.g., a metal or metal salt, such as CuCl₂ (Sigma-Aldrich Cat. No. 203149)) to provide a suitable final concentration (e.g., approximately 8 mM CuCl₂) after mixing. Generally the final concentration of the metal ion, metal, or metal salt is approximately 8 mM but this may be determined empirically according to the sample type and the individual lysis solution. A stock metal ion/guanidinium composition may also be prepared as described above and used for at least about one week. To the metal ion/guanidinium composition may be added a sample (or to the sample may be added the metal ion/guanidinium composition) such as tissue (e.g., rat liver) and the tissue homogenised using standard techniques (e.g., using the QIAGEN RNeasy Mini Kit, Cat. No. 74106, according to manufacturer's instructions; the kits of Table 1). There is no particular limitation to the type of kit used except that it should contain a chaotrope, which may be guanidine. Other tissue and cell types such as, for example, liver, spleen, brain, muscle, heart, oesophagus, testis, ovaries, thymus, kidneys, skin, intestine, pancreas, adrenal glands, lungs, bone marrow or cells such as COS-7, NIH/3T3, HeLa, 293, CHO cells, liquid samples (e.g., serum, plasma, blood) may also serve as the sample The RNA may then be purified immediately from the homogenate using standard techniques or it may be stored for a suitable period of time (e.g., 1 or 8 days) at a suitable temperature (e.g., −70° C., −20° C., 4° C., or 37° C.) before purifying the RNA. The yield and purity of the RNA may then be determined by OD 260/280 nm and the integrity of the RNA determined (e.g., by Q-RT-PCR using oligo dT cDNA priming and β-actin PCR primers (Quantitect SYBR green, QIAGEN) and a Lightcycler (Roche)). Alternatively the metal ion, metal, or metal salt can be added to the tissue lysate immediately after tissue homogenisation in a guanidinium buffer but before storage and purification. Some buffers contain or are suggested by the manufacturer to function optimally in the presence of β-mercaptoethanol, but it may alternatively not be used or substituted by another agent such as, for example, DTT or TCEP.

Certain of these methods may result in the improvement of the stability of a biomolecule (e.g., RNA) within a sample. For example, as shown in Example 1, the stability of β-actin mRNA as determined by Q-RT-PCR following storage of a lysed rat liver sample for 1 or 8 days at 37° C. was improved by approximately 25% following 1 day of storage (e.g., from 38.4% (control, no CuCl₂) to 48.6% (8.2 mM CuCl₂) and by 500% following 8 days of storage (e.g., 4.2% (control, no CuCl₂) to 22.3% (8.2 mM CuCl₂)). Methods for measuring RNA stability are not limited to Q-RT-PCR; any method as is known in the art may be used. As shown in Example 1, the inclusion of about 8 mM metal ion into a biological sample may improve stability of RNA from approximately 25% following storage for 1 day to approximately 500% following storage for 8 days. Thus, the reagents, compositions, and methods described herein improve stability of a biomolecule in a sample stored with a suitable concentration of a metal ion and chaotrope by about 25%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, about 500%, about 525%, about 550%, about 575%, about 600%, about 625%, about 650%, about 675%, about 700%, about 725%, or about 750% as compared to a control stored without the metal ion. In some aspects, the improvement in stability of a biomolecule stored in the presence of the metal ion as compared to that of a biomolecule stored without the metal ion may range from about 25 to about 50%, about 50 to about 75%, about 75 to about 100%, about 100 to about 125%, about 125 to about 150%, about 150 to about 175%, about 175 to about 200%, about 200 to about 225%, about 225 to about 250%, about 250 to about 275%, about 275 to about 300%, about 300 to about 325%, about 325 to about 350%, about 350 to about 375%, about 375 to about 400%, about 400 to about 425%, about 425 to about 450%, about 450 to about 475%, about 475 to about 500%, about 500 to about 525%, about 525 to about 550%, about 550 to about 575%, about 575 to about 600%, about 600 to about 625%, about 625 to about 650%, about 650 to about 675%, about 675 to about 700%, about 700 to about 725%, about 725 to about 750%. The improvement in stability is typically dependent upon the time frame. Methods by which degradation of RNA (e.g., an improvement in stability) is reduced by from about 5% to about 98% (e.g., from about 5% to about 95%, from about 10% to about 95%, from about 20% to about 95%, from about 30% to about 95%, from about 35% to about 95%, from about 40% to about 95%, from about 45% to about 95%, from about 50% to about 95%, from about 60% to about 95%, from about 65% to about 95%, from about 70% to about 95%, from about 75% to about 95%, from about 80% to about 95%, from about 20% to about 85%, from about 25% to about 85%, from about 30% to about 85%, from about 35% to about 85%, from about 40% to about 85%, or from about 30% to about 75%) are also provided. The improvement in stability or reduction in degradation is typically dependent upon the length of time the biomolecule is stored. For instance, these improvements in stability may be provided for anywhere from about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days. These improvements in stability may be also observed for about 1 to about 2 weeks, about 2 to about 3 weeks, about 3 to 4 week, about 1-2 months, about 2-3 months, about 3-4 months, about 4-5 months, about 5-6 months, or longer. These improvements in stability may be observed at these time intervals for various temperatures such as, for example, about 37° C., about 42° C., about 50° C., about 60° C., about 65° C., about 70° C., about 75° C., about 80° C., about 85° C., about 90° C., about 95° C., or about 100° C. Thus, the invention provides compositions (e.g., mixtures containing RNA, one or more chaotropic agent, and one or more metal ion) and methods which allow for the storage of RNA. A functional feature of compostions and methods of the invention is that it allows, in part, for increased RNA stability as compared to other compositions and methods (e.g., mixtures containing RNA and one or more chaotropic agent but not metal ions at a concentration which allow for substantial inhibition of RNA breakdown). Thus, in a functional aspect, the invention provides compostions and methods which allow for the storage of RNA at 37° C. for eight days with from about 5% to about 50%, about 10% to about 50%, about 15% to about 50%, about 18% to about 50%, about 20% to about 50%, about 22% to about 50%, about 25% to about 50%, about 30% to about 50%, about 5% to about 40%, about 10% to about 40%, about 15% to about 40%, about 20% to about 40%, about 5% to about 30%, about 10% to about 30%, about 15% to about 30%, about 20% to about 30%, about 20% to about 25%, of the total RNA remaining intact (e.g., as measured by Q-RT-PCR or other suitable method). Improvements in stability may also be observed during other periods of time and at other temperatures, as would be understood by one of skill in the art. Any of these times and temperatures may be combined with any other to provide a period of time during which the stability of the biomolecule is improved to a particular extent. The effective times and temperatures may vary depending on the type and amount of metal ion, chaotrope, biological sample, and/or type of biomolecule.

As shown in Example 1, reagents, compositions, and methods which increase the stability of RNA are provided herein. One measure of effectiveness of compounds of the invention relates to increases in RNA stabilization over other compounds. As an example, compositions and methods are provided that contain RNA, one or more chaotropic agent, and one or more metal ion at a concentration which stabilizes RNA. The effectiveness of the reagents, compositions, and methods for stabilizing RNA (or other biomolecule) may be determined by comparing the stability thereof in different mixtures: 1) a mixture containing mixtures containing RNA, one or more chaotropic agent, and one or more metal ion at a specific concentration (Mixture 1); 2) mixtures containing RNA, one or more chaotropic agent, and one or more metal ion at a lower concentration lower than Mixture 1 (Mixture 2); 3) mixtures containing RNA, one or more chaotropic agent, and one or more metal ion at a higher concentration lower than Mixture 1 (Mixture 3); 4, 5, 6) mixtures using the concentrations of 1), 2), or 3) but different metal ions (Mixtures 4, 5, 6, respectively); 7) mixtures containing RNA and one or more chaotropic agent (e.g., Mixture 7, the control mixture). The ratio of RNA stabilization seen with Mixture 1 over another Mixture (e.g., Mixtures 2-7) may be in the ranges of from about 20 to about 1, from about 15 to about 1, from about 12 to about 1, from about 10 to about 1, from about 9 to about 1, from about 8 to about 1, from about 7 to about 1, from about 6 to about 1, from about 5 to about 1, from about 4 to about 1, from about 3 to about 1, or from about 2 to about 1. As described above, the stability of the RNA may be determined using any suitable method available to one of skill in the art (e.g., Q-RT-PCR as shown in Example 1).

Certain biological samples are provided with EDTA. In such cases, it may be necessary to calibrate the amount of metal ion (e.g., CuCl₂, FeCl₃) necessary to saturate the EDTA chelator and obtain a suitable final concentration of metal ion (e.g., about 6 to about 8 mM). This may be accomplished essentially as described in Examples 6 and 8 below. As shown therein, the concentration of EDTA within an RNA-containing sample must be kept below an amount harmful to RNA (e.g., 8 mM). Thus, in one aspect, this disclosure provides a method for stabilizing RNA using a composition comprising a chaotrope (e.g., guanidine, arginine) that comprises either less than 8 mM EDTA, or does not also comprise EDTA. In other aspects, methods are provided for inhibiting and/or preventing the deleterious effects of EDTA on RNA during storage by introducing a suitable concentration of a metal ion into the RNA-containing composition.

Even in the absence of RNases, pure RNA in pure guanidine is significantly more stable than pure RNA in water, the addition of a metal ion, metal, or metal salt in a suitable concentration (e.g., 8 mM CuCl₂) to a chaotropic solution containing or use to isolate RNA may be used to stabilize the sample even better than the chaotrope alone. The RNA may then be stored and transported at room temperature or on ice rather than frozen on dry ice.

Certain manufacturer's protocols require heating the RNA-containing guanidine lysate at 70° C. for 3 minutes or more which is known to negatively impacts the quality of the RNA. As described in Example 9, it has been found that the addition of a suitable concentration of metal ion (e.g., 8.2 mM CuCl₂) minimises degradation during such steps. In fact, it has been found that, in the presence of a suitable concentration of metal ion (e.g., 8 mM CuCl₂), it is possible to increase such incubation steps to 5 minutes or more at 70° C. As such, one may increase RNA yields from tissues that are known to be difficult to lyse (e.g., skeletal muscle, heart tissue, skin tissue, and/or other tissue rich in structural proteins such as collagen, actin, myosin, keratin, and/or elastin). Thus, the reagents, compositions, and method provide for the incubation of RNA within a tissue lysate at high temperature (e.g., about 37° C., about 42° C., about 50° C., about 60° C., about 65° C., about 70° C., about 75° C., or about 80° C., about 85° C., about 90° C., about 95° C., or about 100° C.) for up to about 3, about 4, about 5, about 6, about 7, about 8, about 9, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, or about 100 minutes without significant degradation of RNA. The skilled artisan would understand that any suitable concentration of metal ion could be used, and that the time and temperature of the incubation step may be altered as necessary.

Commonly, mechanical means may be used to disrupt samples during processing. For instance, TissueRuptor® or TissueLyser II® (QIAGEN), OMNI Ruptor® (OMNI International, a Precellys®24 (BERTIN Technologies), Polytron (Brinkmann), Tekmar Tissuemizer® (Tekmar Tissuemizer Co), or Omni-Mixer® (SORVALL) may be used. Unfortunately, such mechanical methods, especially when ceramic or steel beads are used, may facilitate disruption of the cells and tissues within the sample but also lead to friction and significant heating of the sample. Heating the lysate in the presence of guanidine leads to rapid degradation of the RNA. In order to reduce such unwanted RNA degradation, the extent and intensity of the mechanical disruption must be limited and sometimes additional cooling systems must be used. However, it has been found that the inclusion of a suitable concentration of metal ion, metal, or metal salt in addition to a chaotrope provides the skilled artisan the ability to increase the intensity of the mechanical disruption (thereby allowing shorter run times and higher sample throughput) without negatively affecting the integrity of the biomolecule (e.g., RNA).

In one aspect, a kit for processing a biomolecule (e.g., extracting it from a sample, such as a biological sample), which kit comprises a source of metal ions as described herein and optionally a chaotrope as described herein (e.g., guanidine). The source of metal ions may be, for example, a stock solution of metal ions that may be diluted into a chaotrope or a sample to provide a suitable concentration of metal ions. The chaotrope may be provided as a separate composition (at a suitable concentration amenable to dilution to an effective amount in the sample). The metal ions may also be provided as a mixture with the chaotrope at a ratio (as described herein) that is suitable for use with a sample. It is preferred that the kit comprise a mixture of metal ions and chaotrope at a concentration and a ratio to one another that is suitable for dilution to an effective amount in a sample. The kit may also include instructions for contacting the sample with the metal ion and/or chaotrope (e.g., guanidine) so as to improve processing of the biomolecule (e.g., adding the metal ion and chaotrope as a step in the isolation of the biomolecule). The biomolecule may be RNA isolated from a biological sample. In a further aspect, the present invention provides a kit for extracting RNA from a biological sample, which kit comprises guanidine and a source of metal ions for mixing with the sample, wherein the metal ion is derived from a metal other than from a Group 1 or Group 2 metal of the Periodic Table, the kit further comprising instructions for contacting the sample with the guanidine and the metal ion so as to provide a metal ion concentration of no more than 20 mM whereby the RNA is stabilised against degradation, wherein the metal ion is derived from a metal other than from a Group 1 or Group 2 metal of the Periodic Table. In a further aspect, a kit for extracting a biomolecule (e.g., RNA) from a biological sample is provided. The kit typically comprises a source of metal ions and, in the same or a separate composition, guanidine that, when introduced into the biological sample together or separately, provide a concentration chaotrope and metal ions such that the processing of the biomolecule is improved (e.g., increased stability, less degradation thereof). The reagents, compositions and methods provided herein may also be used in addition to or as a substitute to wash solutions provided in commercially available kits, for example. Other embodiments of such kits are also possible as would be understood by one of skill in the art.

In a further aspect, the present invention provides use of metal ions derived from a metal other than from a Group 1 or Group 2 metal for stabilising RNA in a sample in the presence of guanidine. In one aspect, a composition comprising a combination of a source of metal ions for mixing with the sample, and optionally a chaotrope such as guanidine, that may be used to improve processing of a biomolecule (e.g., extracting it from a biological sample) by mixing the composition with the sample before or during processing is provided. In some aspects, the biomolecule is RNA which is to be isolated from a biological sample. In a further aspect, the present invention provides use of a combination of guanidine and a source of metal ions for stabilising RNA during extraction of the RNA from a biological sample, wherein the guanidine and metal ion are contacted with the sample to form a stabilised RNA-containing composition in which the metal ion is present at a concentration of no more than 20 mM, and wherein the metal ion is derived from a metal other from a Group 1 or Group 2 metal. In other aspects, the final metal ion concentration in the sample is less than about 20 mM; less than about 10 mM; about 8 mM; or 8.2 mM. In other aspects, a composition for extracting RNA from a biological sample, typically comprising guanidine and a concentration of about 8.2 mM copper (e.g., Cu²⁺), is provided. Other concentrations of metal ions may also be suitable, alone or in combination with a chaotrope such as guanidinium, as would be understood by one of skill in the art.

In one aspect, a stabilized biomolecule-containing composition comprising a combination of a source of metal ions for mixing with the sample, and optionally a chaotrope such as guanidine, is provided. In some aspects, the biomolecule is RNA isolated from a biological sample. In a further aspect, the present invention provides a stabilised RNA-containing composition, which comprises RNA, guanidine and a metal ion, wherein the metal ion is present at a concentration which is no more than 20 mM and the metal ion is derived from a metal other than from a Group 1 or Group 2 metal of the Periodic Table. In other aspects, the metal ion concentration in the stabilised RNA-containing composition is less than about 20 mM; less than about 10 mM; about 8 mM; or 8.2 mM; whereby the RNA is stabilised against degradation. In other aspects, stabilised RNA-containing composition comprises guanidine and a concentration of about 8.2 mM copper (e.g., Cu²⁺) or iron (e.g., or Fe³⁺) is provided. Other concentrations of metal ions may also be suitable, alone or in combination with a chaotrope such as guanidinium, as would be understood by one of skill in the art.

A stabilised RNA-containing composition may be defined as a crude mixture derived from (i) a biological sample such as serum, cells or tissue containing RNA, (ii) a chaotrope such as a guanidine salt in the final concentration range of about 200 mM to about 10M, and (iii) one or more metal ions in the final concentration range about 0.1 to about 50 mM.

Provided herein are compositions comprising a combination of guanidine and a sufficiently low concentration of certain metal ions for stabilising biomolecules against degradation. It has surprisingly been found that a combination of guanidine with a sufficiently low concentration of certain metal ions is capable of stabilising RNA against degradation. This is surprising because the use of guanidine during the lysis of biological samples has been found to generate a reactive composition which leads to the rapid degradation of some biomolecules, in particular, RNA. Equally the presence of metal ions have hitherto been demonstrated to be undesirable for RNA, leading to the widespread use of chelators to limit RNA degradation by removing the metal ions from solution. The sufficiently low concentration of metal ion required to stabilise RNA against degradation may be any suitable concentration or range of concentrations as described herein. The sufficiently low concentration of metal ion required to stabilise RNA against degradation may also be, for example, less than about 20 mM; less than about 10 mM; about 8 mM; or 8.2 mM. In other aspects, sufficiently low concentration of metal ion to stabilise the RNA against degradation is about 8.2 mM copper (e.g., Cu²⁺). Other sufficiently low concentrations may also be suitable as would be understood by one of skill in the art.

It is believed that amines may contribute to the degradation of biomolecules during processing. The reagents, compositions, and methods described herein are, in some aspects, designed to prevent degradation of biomolecules by amines. As discussed herein and without wishing to be bound by theory, it is thought that during typical lysis steps involved in the extraction of RNA from biological samples amines are released which may contribute to the degradation of RNA. This degradation effect is thought to be abrogated by the combined presence of metal ions and guanidine according to the present invention. Compounding the problem is the extremely effective chaotropic property of guanidine so that its use results in a correspondingly large amount of amines being released from the sample. Consequently the greater release of amines results in a larger amount of the reactive composition being formed leading to greater biomolecule degradation. Sources of cellular or non-cellular biological amines include but are not limited to the α-amino group (NH2-) of amino acids, peptides and proteins. The reactive composition can be formed from mixtures of guanidine and lysed bacterial or eukaryotic cells, tissues, blood, pure proteins such as BSA and immunoglobulins, non-cellular biological samples such as serum, plasma, saliva, urine, CSF and tissue culture medium, or extracts from biological or non-biological samples such as forensic samples or synthetic materials that have potentially been in contact with biowarfare agents such as soil, clothing or skin. Provided herein are reagents, compositions and methods for inhibiting, preventing, or affecting the degradation of biomolecules by amines during processing. A suitable concentration of metal ions may be any concentration of metal ions, either alone or in combination with another agent such as a chaotrope (e.g., guanidine), that inhibits, prevents, or affects the ability of amines to contribute to the degradation of biomolecules by amines during processing. The metal ion concentration required to stabilise RNA against degradation by amines may be any suitable concentration or range of concentrations as described herein. For example, the metal ion concentration used to inhibit and/or prevent degradation of RNA by amines during processing of samples may be less than about 20 mM; less than about 10 mM; about 8 mM; or 8.2 mM. For instance, a concentration of about 8.2 mM copper (e.g., Cu²⁺) may be used. Guanidine is typically the chaotrope of choice to prevent degradation of biomolecules by amines. Other metal ion concentrations that inhibit, prevent, or affect the ability of amines to contribute to the degradation of biomolecules may also be suitable as would be understood by one of skill in the art.

The invention is not particularly limited to any one type of biomolecule, but the improvement of the quality and integrity of RNA is particularly notable. The invention can also be used to lyse and stabilise samples for the analysis of miRNA, sRNA and other small naturally occurring RNA molecules such as snRNAs, snoRNA, ncRNA, snoRNA, piRNA and rasiRNA. It can also be used for studies, diagnostics and therapies involving synthetic RNA of the RNAi type. Usefully the invention can be used to preserve viral RNA such as retroviruses e.g. HIV, rotaviruses e.g. HCV, and West Nile Virus in mixtures of guanidine and blood, serum, plasma cells and/or other medically important sample types such as cells and tissues. The RNA referred to can be found, derived or associated with a sample such as a virus, cell, serum, plasma, blood, BAL, Ascites and CSF preserved samples such as FFPE blocks or sections, biopsies and solid or liquid tissues. It can also be a nucleoside or nucleotide containing molecule such as a cAMP, ATP, GTP, monomers, dimers and oligomers of deoxy- and ribonucleotides, deoxy- or ribo-oligonucleotides, plasmid DNA, genomic DNA, mitochondrial DNA, RNA such as microRNA (miRNA), piRNA, sRNA, tRNA, viriods, hnRNA, mRNA, rRNA such as the 5S, 5.8S, 16S, 18S, 23S and 28S rRNA species, and viral RNA derived from for example HCV, West Nile Disease Virus, Foot and Mouth Disease Virus, Influenza, SARS, or HIV RNA and be single (ssRNA) or double stranded RNA (dsRNA) or hybridised RNA and DNA (dsRNA:DNA) or a mixture of single and double stranded sequences. Usefully the invention can be used to lyse and preserve single and/or double stranded RNA (ssRNA and dsRNA) viruses including animal RNA viruses such as Norwalk, Rotavirus, Poliovirus, Ebola virus, Marburg virus, Lassa virus, Hantavirus, Rabies, Influenza, Yellow fever virus, Corona Virus, SARS, West Nile virus, Hepatitis A, C(HCV) and E virus, Dengue fever virus, toga (e.g. Rubella), Rhabdo (e.g. Rabies and VSV), Picorna (Polio and Rhinovirus), Myxo (e.g. influenza), retro (e.g. HIV, HTLV), bunya, corona and reoviruses which have profound affects on human health including viroid like viruses such as hepatitis D virus and plant RNA viruses and viroids such as Tobus-, Luteo-, Tobamo-, Potex-, Tobra-, Como-, Nepo-, Almo-, Cucumo-, Bromo-, Ilar-viruses, Coconut cadang-cadang viroid and potato spindle tuber viroid which all have profound effects on agricultural production are all liable to be degraded before, during or after extraction for diagnostic detection purposes. The invention can also be used for lysing and stabilising single stranded RNA bacteriophage such as the genus Levivirus including the Enterobacteria phage MS2 and the genus Allolevivirus including the Enterobacteria phage Qβ, or double stranded RNA bacteriophage such as Cystovirus including Pseudomonas phage φ₆ or other types of phage such as those used as internal RNA controls for diagnostic applications such as those used in Armored RNA® (Ambion). The quality, stability, and/or integrity other types of biomolecules may also be improved using the compositions and methods described herein as would be understood by one of skill in the art.

Branched DNA or bDNA assays commercialised as VERSANT bDNA 3.0 Assay™ (Bayer Corp) are commonly used to determine HCV and HIV blood titres. Advantageously RNA purification is not necessary, rather the bDNA assay relies on hybridisation of the probe to the target RNA in the lysed solution containing guanidine. However, there are two separate heating steps that most likely lead to RNA degradation and therefore reduced assay sensitivity or variation. These are a 2 hour (HIV VERSANT) or 1 hour (HCV VERSANT) viral lysis step at 63° C. followed by an extended hybridisation reaction at 16 to 18 h at 52° C. (HIV VERSANT) or 15 to 17 h at 53° C. (HCV VERSANT). Heating the analyte HIV or HCV RNA such high temperatures for such extended times in the presence of guanidine will result in RNA degradation as has been suggested by Elbeik et al., J. of Clin. Microbiol. (2004) 42:3120. By using mixtures of guanidine and metal salts RNA degradation is reduced during the lysis and hybridisation steps of the bDNA assay thereby improving detection and decreasing variation.

It can also be a molecule derived from a synthetic organic procedure such as an oligo-synthesizer, a mixture of RNA and DNA, a chimera of RNA and DNA, the product of an enzymatic reaction such as an in vitro RNA transcription, amplified RNA (aRNA), ribozymes, aptamers, a PCR amplification, rolling circle amplification (RCA) or ligase chain reaction (LCR) an internal control standard or control RNA. The quality, stability, and/or integrity other types of molecules derived from a synthetic organic procedure may also be improved using the compositions and methods described herein as would be understood by one of skill in the art.

RNA analysis methods that would benefit from this invention include in vitro or in vivo protein translation of mRNA templates, RNA dependent RNA polymerisation, DNA dependent RNA polymerisation, RNA splice analysis, RNA folding analysis, aptamer and ribozyme production, optical density (OD) measurements, RNA:protein interaction studies, RNA electrophoresis and sedimentation including molecular weight standards, RNA bioconjugates, RNA ligation, RNA folding studies, RNA footprinting, RNA NMR structural studies, RNA oligonucleotide synthesis, RNA in situ, RNA sequencing, reverse transcription (RT), RT-PCR, Q-RT-PCR, nuclease protection assays, hybridisation techniques such as Northern blotting, bDNA, and microarrays including the preparation of probes, fluorescent nucleic acid labelling, NASBA, RNAi, miRNA techniques such as extraction and quantification and those methods requiring quality control and/or quantitative or qualitative measurements of RNA. Other types of RNA analysis methods may also benefit from using the compositions and methods described herein as would be understood by one of skill in the art.

Instability refers to an alteration in the molecular weight or an alteration of the chemical structure of the RNA molecule, such instability is associated with handling, storage, transport and/or the actual analysis of the analyte molecule. Biomolecule instability is often related to the activity of naturally occurring catabolic enzymes and in particular RNases which can substantially alter the molecular weight of the RNA or involve much smaller molecular weight alterations of the original analyte molecule. Such RNases can have an origin either in the biological sample itself, for example they can be released progressively following sample handling or released massively as a result of poor handling of the tissue when for example it has been freeze thawed, a process that generally leads to the rupture of intra-cellular vesicles containing proteases and nucleases that consequently flood into the cytoplasm leading to very high rates of analyte degradation. Alternatively, the degradative enzyme can come from external contamination of the sample environment such as microbial contamination or spoilage of the sample. A suitable concentration of metal ions may be any concentration of metal ions, either alone or in combination with another agent such as a chaotrope (e.g., guanidine), that inhibits, prevents, or otherwise positively affects instability of RNA during processing. Suitable metal ion concentrations that may be any suitable concentration or range of concentrations as described herein. Other metal ion concentrations that inhibit, prevent, and/or otherwise positively affects instability of a biomolecule such as RNA during processing may also be suitable as would be understood by one of skill in the art.

Analyte instability is generally associated with a reduction in the sensitivity or performance of the analytical procedure, whether the analyte is a nucleic acid, oligo-ribonucleotide and oligo-deoxyribonucleotide.

‘Degradation’ refers to the physical or chemical changes that occur as a consequence of biomolecule/analyte instability. As some examples of degradation related to nucleic acids, degradation can refer to the deamination of nucleobases such as the conversion of cytosine to uracil, the loss of methyl groups from methyl-cytosine, the loss of one or more nucleobases such as occurs during depurination, the cleavage of phosphodiester bonds leading to chain cleavage and the loss of one or more nucleotides from the bulk of the nucleic acid molecule. It does not refer only to changes of the secondary or tertiary structure of the molecule. A suitable concentration of metal ions may be any concentration of metal ions, either alone or in combination with another agent such as a chaotrope (e.g., guanidine), that inhibits and/or prevents degradation of RNA during isolation, handling, storage, transport and/or analysis. Suitable metal ion concentrations that may be used for inhibiting and/or preventing degradation of a biomolecule such as RNA during processing may any suitable concentration or range of concentrations as described herein. Other metal ion concentrations may also be suitable for inhibiting and/or preventing degradation as would be understood by one of skill in the art.

‘Integrity’ refers to the intactness of a molecule and therefore is the opposite of degradation. A suitable concentration of metal ions may be any concentration of metal ions, either alone or in combination with another agent such as a chaotrope (e.g., guanidine), that inhibits and/or prevents loss of and/or maintains the integrity of RNA during isolation, handling, storage, transport and/or analysis. Suitable metal ion concentrations that may be used for inhibiting and/or preventing loss of and/or maintaining the integrity of a biomolecule such as RNA during processing may be any suitable concentration or range of concentrations as described herein. Other metal ion concentrations may also be suitable for inhibiting and/or preventing a loss of and/or maintaining the integrity of the biomolecule such as RNA during processing as would be understood by one of skill in the art.

‘Substantial degradation’ refers to a sample that contains at least half of the analyte molecules that have been cleaved or reduced in molecular weight by 5% or more. A suitable concentration of metal ions may be any concentration of metal ions, either alone or in combination with another agent such as a chaotrope (e.g., guanidine), that inhibits and/or prevents substantial degradation of a biomolecule such as RNA during processing. Suitable metal ion concentrations that may be used to inhibit substantial degradation of as such may include any suitable concentration or range of concentrations as described herein. Other metal ion concentrations may also be suitable for inhibiting and/or preventing substantial degradation of a biomolecule such as RNA during processing as would be understood by one of skill in the art. Methods to determine degradation are well known and depend on the analyte molecule (see Sambrook et al., (1989) Molecular Cloning: A Laboratory Manual (2nd Ed.) Cold Spring Harbor University Press, NY). The determination of the molecular weight and therefore the extent of degradation of nucleic acids is commonly carried out using denaturing or native gel electrophoresis and may include Southern or Northern blotting with a labelled hybridisation probe. RNA quantification can include analysis using an RNA Chip and the Agilent Bioanalyser 2100™ system and calculating the ‘RNA Integrity Number’ (RIN). Nucleic acid degradation can also be conveniently quantified by Q-PCR for DNA, and Q-RT-PCR for RNA using for example a Lightcycler™ (Roche) and suitable amplification probes. Calculating the Q-RT-PCR amplification ratios of 3′/5′ ends of a mRNA, frequently β-actin, following reverse transcription using an oligo dT primer is also commonly used to assess RNA degradation. Other methods include comparing the relative hybridisation signals of oligonucleotides representing 3′ to 5′ sites of mRNA following analysis using Affymetrix® GeneChips®. Smaller single or double stranded nucleic acids of less than 100 nucleotides in length such as oligonucleotides and miRNA are most accurately quantified by mass spectrometry such as MALDI-TOF MS, this technique having the added advantage of being able to also determine degradation events that do not significantly alter the molecular weight of the analyte such as depurination or deamination of nucleobases. Most miRNA analyses are carried out by dedicated Q-RT-PCR. Despite the sophistication of the methods for determining the extent of RNA degradation, it is evident that certain mRNA are far more sensitive to degradation than others and because the 18S and 28S rRNA are relatively stable to degradation, they are only a poor surrogate marker for the extent of mRNA degradation. Accurately analysing mRNA degradation is currently best carried out using Q-RT-PCR as explained above. For a detailed description of methods to evaluate RNA degradation as a result of RNA purification methods see Muyal et al., (2009) Diag. Path. 4:9.

Determining empirically the most appropriate type and amount of metal or metal salt to be used according to this invention requires a comparison of the RNA yield, the RNA purity by OD 260/280 measurements and RNA integrity after storage and/or purification as set out above using gel analysis, RIN determination and Q-RT-PCR. It will be understood however that there are other methods to determine the appropriate amount and type of metal or metal salt that should be added to the lysis solution such as using hybridisation of bDNA probes directly in the lysate, cDNA or aRNA probes and microarrays (e.g. Affymetrix, Agilent). It may also be necessary to compare specific target RNA types or identities such as miRNA, mRNA, rRNA or viral RNA, and ssRNA to dsRNA. Such comparisons can only reliably be carried out empirically.

A pure sample of a nucleic acid (e.g., RNA or DNA) refers to solution thereof in water where the OD260/280 ratio is 1.7 or above. A pure sample should have a OD 260/230 nm ratio of at least about 1.6, whilst absorption at wavelengths greater than 330 nm indicates large particles are contaminating the sample. A pure sample should have a absorption at 330 nm or greater of zero. Some contaminants in an otherwise pure sample may not be determined by absorption spectrometry, rather these may be detected by another technique (e.g., mass spectrometry such MALDI-TOF, or by observing an inhibition of the RT reaction as determined by Q-RT-PCR). A suitable concentration of metal ions may be any concentration of metal ions, either alone or in combination with another agent such as a chaotrope (e.g., guanidine), that may be used to provide and/or maintain a pure sample of RNA (e.g., during extraction, isolation, handling, storage, transport, and/or analysis thereof). A suitable metal ion concentrations that may be used to provide and/or maintain a pure sample of a biomolecule such as RNA may be any suitable concentration or range of concentrations as described herein. Other metal ion concentrations may also be suitable to provide and/or maintain a pure sample of a biomolecule such as RNA during processing as would be understood by one of skill in the art.

Although generally instability and degradation are associated with a reduction in the overall molecular weight of the molecule under study (“the analyte”), it can, conversely, be related to an increase in the molecular weight of a complex that progressively aggregates during, for example, storage. One example of the latter would be the complexation or aggregation of proteins onto nucleic acids during storage of a whole tissue or the chemical cross-linking of molecules during the processing of a sample such as with formalin fixed paraffin embedded tissue (“FFPE”). A suitable concentration of metal ions may be any concentration of metal ions, either alone or in combination with another agent such as a chaotrope (e.g., guanidine), that may be used to preventing and/or inhibiting an increase or decrease in the molecular weight of a biomolecule such as RNA during processing. Exemplary, non-limiting, and suitable metal ion concentrations that may be used to prevent and/or inhibit an increase or decrease in the molecular weight of the biomolecule such as RNA during processing may include, for example, any suitable concentration or range of concentrations as described herein. Other metal ion concentrations may also be suitable for preventing and/or inhibiting an increase or decrease in the molecular weight of the biomolecule such as RNA during processing as would be understood by one of skill in the art.

Stabilisation refers to conditions that lead to an overall reduction in the amount of degradation of an analyte molecule compared with the control. Such a control is commonly the conditions used without the use of the invention. The control may be an excised piece of tissue such as a biopsy, a blood or serum sample or a piece of tissue lysed in a pure 5M solution of Guanidine HCl pH 7.0 at for example 4, 20 or 37° C. In such embodiments, an analyte (e.g., RNA) may be stabilized by reducing the amount of degradation of the analyte as compared with the control.

Biomolecule extraction and purification can generally be divided into two stages; 1) sample homogenisation and lysis, 2) differential purification of different classes of biomolecules from one another. The specific type of lysis depends on the sample type and the final analytical procedure. For example fibrous tissues such as skin, heart or lignified plant material requires substantial physical grinding for effective homogenisation whilst tissue culture cells can often be lysed by simply adding a chaotropic salt. Differential purification is the process of removing, for example proteins from nucleic acids, and RNA from DNA and vice versa.

The sample containing the analyte can be a (i) liquid sample such as blood, plasma, serum, cerebral spinal fluid (CSF), sputum, semen, bronchoalveolar lavage (BAL), amniotic fluid, milk and urine, (ii) solid samples such as body tissues (liver, spleen, brain, muscle, heart, oesophagus, testis, ovaries, thymus, kidneys, skin, intestine, pancreas, adrenal glands, lungs, bone and bone marrow), (iii) clinical samples for a medical test such as a prostate, breast or a cancer sample, tumour or biopsy, including a FFPE sample, blood test, clinical swabs, dried blood, (iv) animal tissues derived from biomedical research or fundamental biology (monkey, rat, mouse, Zebra fish, Xenopus, Drosophila, nematode, yeast) and from their various stages of development (egg, embryo, larvae, adult), (v) tissue and tissue culture cells used for drug discovery purposes, (vi) pathogenic and non-pathogenic microbes such as fungi, archaebacteria, gram-positive and gram-negative bacteria, including E. coli, Staphylococcus, Streptococcus, Mycobacterium, Pseudomonas and bacteria that cause Shigella, Diphtheria, Tetanus, Syphilis, Chlamydia, Legionella, Listeria and leprosy, (vii) pathogenic or non-pathogenic viroids, bacteriophage or viruses that are found in a variety of biological samples such as bacteria, plants, blood, human tissues, animals blood, seum, plasma and tissues, and clinical samples, (viii) plants such as the leaves, flowers, pollen, seeds, stems and roots of rice, maize, sorghum, palm, vines, tomato, wheat, barley, tobacco, sugar cane and Arabidopsis, (ix) fixed tissue such as FFPE tissues and biopsies which frequently require specialised protocols for extracting high quality nucleic acids, (x) potentially pathogenic material associated with bioterrorism threats such as anthrax that may or may not need to be transported from the discovery site to the testing facility, (xi) extremely small samples such as those derived from Laser Capture Microdissection samples (LCM), (xii) food samples that may for example contain food borne diseases, (xiii) soil sample. It should be noted that the sample may not be derived solely from biologically derived samples but also chemically or enzymatically synthesised ones such as nucleic acid based copied molecules or amplification products such as in vitro transcribed RNA and PCR products, oligodeoxyribonucleotides and oligoribonucleotides, PNA and LNA. There is no particular limitation to the type of sample that can be used with this invention. Such samples may also be biological samples, and are typically RNA-containing samples.

The invention is also useful for the stabilisation of RNA internal controls (IC) and standards such as those included in HIV or HCV diagnostic kits such as Amplicor™ (Roche) or for carrier RNA that can be included in such diagnostic kits. For this use, the RNA IC is commonly transported and stored with the rest of the kit components, often at room temperature or 4° C. which may lead to degradation. Stabilisation of the RNA IC or carrier RNA improves kit performance and maintains its integrity during transport and storage. In such embodiments, the RNA may be stabilised as described herein.

It is well known that not only are salts of guanidine extremely effective at dissociating and disrupting biological samples, but they also usefully destroy the tertiary structure of most proteins including catabolic enzymes such as RNases. Despite the widespread use of guanidine salts in home-brew and commercilised kits their application for preserving biomolecules is significantly limited because in the presence of the amines that are released from nearly all biological samples during lysis, a reactive composition is formed which leads to the rapid degradation of some biomolecules but in particular RNA. In certain embodiments, the compositions and methods described herein (e.g., containing and/or using metal ions and a chaotrope such as guanidinium) inhibit and/or prevent the formation of such reactive compositions.

One of the distinct advantages of this invention is that both sample lysis and stabilisation take place in the same mixture, so that it is that it is possible to stabilise the RNA analyte in the lysed sample and then subsequently purify the intact nucleic acid from the same solution thereby increasing yields and improving throughput for example for viral diagnostic applications. Therefore instead of two reagents being necessary; a stabilisation reagent and a lysis reagent, according to this invention, only one combined stabilisation and lysis reagent is necessary thereby simplifying the protocol, the number of reagents required in the kit, the potential for contamination and increasing the sensitivity and simplicity of the test. Advantageously according to this invention, the RNA in this lysing and stabilising solution, can in the presence of an appropriate amount of alcohol be made to bind to solid phases in particular silica with no substantial loss of yield compared with standard methods. Alcohol (e.g., generally one volume lysate (sample plus lysis buffer) to one volume 50-70% ethanol) may be added in order to enhance biomolecule (e.g., RNA) binding to, for instance, a silica membrane. Other aspects of such embodiments are also contemplated as would be readily apparent to the skilled artisan.

Preferably when additional metal ions are added to the lysis solution, the amount of DNA, protein and other contaminants binding to the solid phase is reduced whilst the amount of RNA binding is either not altered or is increased. It is also preferred that the distribution and size of the RNA molecules that are purified are not significantly altered, or the small RNA species such as miRNA are co-purified with the larger RNA species such as rRNA and mRNA, or the relative binding of miRNA compared with larger RNA can be controlled in a reproducible manner by varying salt and ethanol concentration in the binding mix. In some embodiments, the “RNA” may include one, more than one, or all of such RNAs, and the RNA may be considered stabilised when it maintains one, some, or all of these properties. This invention therefore relates to methods to improve the storage, preservation, archiving, transport, extraction and purification, to protect RNA from degradation, to increase its stability and as a consequence, to improve the analytical sensitivity and assay quality. Because salts of guanidine are very widely used chaotropes for the lysis, homogenisation, dispersion and release of biomolecules from biological samples such as blood, serum, plasma and tissue this invention will find widespread application.

It has been found that the amount of RNA degradation occurring in guanidine mixtures of biological samples is very sensitive to: (i) the duration of the incubation, (ii) the temperature, (iii) the concentration of guanidine used, (iv) the presence of metal ions or salts. It has surprisingly been found that a pure sample of RNA mixed with a pure solution of guanidine is quite stable, indeed significantly more stable than RNA in water alone, but unexpectedly, when biological material such as cells, tissue, serum or plasma is added to the guanidine/RNA mixture, rapid and substantial RNA degradation occurs. It has been determined that RNA degradation is not due to RNase activity, rather it is dependent on the presence of amines. Notably, the concentration of amino acids and other amines in the sample has been found to be a crucial determinant of the extent of RNA degradation in the guanidine solution. As one example, the concentration of a single amino-acid, lysine, in endothelial cells can reach 2 mM (Loscalzo et al., (2001) J. Clin. Invest. 108:663) and in plasma 0.1 mM (Creager et al., J. Clin. Invest. 90:1248) and it would be expected that the total amino-acid (amine) content in the cell would be be much greater. Primary and secondary amine concentrations can be determined by a variety of methods including the well-known colourimetric Ninhydrin assay. This assay may be used to simply determine the reduction in free amine in a sample following treatment with a metal ion or salt, that is what proportion of the amines complex or are chelated by the metal ion or salt and therefore cannot participate in RNA degradation (Example 17). To carry out the this assay, Ninhydrin (e.g., 50 mg/ml in water) may be added added to a guanidine RLT buffer (Qiagen) containing homogenised rat liver and mixed. The colour change is typically determined after incubating for 15 minutes at 20° C. by spectrometry at 570 nm. Identical mixtures can be prepared and tested by adding variable amounts and types of metal ions and salts, the reduction in absorbance at 570 nm indicates that amines are being complexed or chelated by the metal ions thereby providing a simple screen to determine potentially useful metal ions and salts and their appropriate concentrations of use. Appropriate final concentrations of metal ions or salts can be tested in the range of 0.1-20 mM, such as 0.1, 0.5, 1, 2, 3, 4, 5, 6, 7, 8, 9, 10, 11, 12, 13, 14, 15, 16, 17, 18, 19, and 20 mM, and more preferably less than 20 mM, 15 mM, 10 mM, or 5 mM. It should be noted that the Ninhydrin test can provide only approximate results and that empirical tests of RNA quality and yield are preferred to determine the optimum use of the metal ion or salt. Alternatively, the concentration of the primary or secondary amine in the biological sample can be determined by preparing a standard curve with known amounts of amine in RLT and comparing with the unknown sample which may help to optimise adding the correct amount of metal ion or salt to samples containing particularly high concentrations of amine.

The relationship between amine or amino-acid type and/or concentration, guanidine and the extent of RNA degradation can conveniently be determined by the method as set out in Example 18. To carry out this assay, a pre-purified source of RNA (e.g., 24 of total rat liver RNA) may be added to a 6M solution of guanidine HCl (e.g., 500) or buffer RLT (Qiagen), varying final concentrations of amines such as ethylenediamine (e.g., 30 μM), Lysine (e.g., 300 μM), Histidine (e.g., 300 μM), glycine (e.g., 300 μM), and/or a protein such as BSA (e.g., 20 μg). The mixture may then be (e.g., at 60 to 70° C. for 30-90 minutes) before purifying the RNA (e.g., using a silica spin-column (QIAprep, Qiagen)) and assessing the extent of degradation (e.g., by agarose gel electrophoresis). Conveniently, the protective effects of adding metal ions (e.g., CuCl₂) can also be determined in the same manner by making the mixture of RNA/guanidine/amine, and then adding a source of metal ions (e.g., CuCl₂) prior to the heating step, purification and RNA analysis. In this manner it is possible to screen for the most appropriate metal ions that provide RNA stability in a guanidine/amine mixture.

Therefore it has been found that although commercial preparations of guanidine lysis buffers such as those set out in Table 1 have been used for many years, they are very poorly adapted to either storage or conditions that increase the temperature of the sample such as during mechanical disruption, these same commercial or home-brew laboratory lysis solutions can be markedly improved by the simple addition of various metal reagents such as copper (II) chloride to the guanidine containing lysis reagent. Following the addition of the metal or metal salt to the guanidine solution it has surprisingly been found that the mixture has a markedly improved capability to stabilise RNA and other biomolecules in solution prior to extraction but does not markedly alter the yield of extraction for either RNA or DNA.

All references cited within this disclosure are hereby incorporated into this disclosure in their entirety. All numerical ranges disclosed herein include each numerical value within each of said ranges as if each such value had been listed individually. Certain embodiments are further described in the following examples. These embodiments are provided as examples only and are not intended to limit the scope of the claims in any way.

### EXAMPLES

### RNA Stabilisation in Animal Tissue Lysates

To 600 μl of Buffer RLT (QIAGEN) containing 6 μl 14.3M β-mercaptoethanol, was added 10 μl of a 0.5M solution of a metal or metal salt such as CuCl₂ (Sigma-Aldrich Cat. No. 203149) to give a final concentration of approximately 8 mM CuCl₂ and briefly mixed by inversion. Alternatively a stock solution of several millilitres can be prepared and used for at least one week. To the guanidine/mercaptoethanol/metal salt mixture was added 4-30 mg of rat liver and the tissue homogenised according to manufacturer's instructions (QIAGEN RNeasy Mini Kit, Cat. No. 74106). 600 μl portions of the lysate were then purified immediately according to manufacturer's instructions or stored for 1 or 8 days at 37° C. before purification according to manufacturer's instructions and elution in 100 μl of water. The yield and purity of the RNA was then compared by OD 260/280 nm and the integrity of the RNA determined by Q-RT-PCR using oligo dT cDNA priming and β-actin PCR primers (Quantitect SYBR green, QIAGEN) and a Lightcycler (Roche).

Alternatively the metal or metal salt can be added to the lysate immediately after tissue homogenisation but before storage and purification. The β-mercaptoethanol can alternatively be deleted from the mixture or be replaced with DTT or TCEP. Other commercialised RNA purification kits can replace the RNeasy kit as set out in Table 1, there is no particular limitation to the type of kit used except it should contain a chaotrope, preferably guanidine. Generally the final concentration of the metal or metal salt added is approximately 8 mM but should be determined empirically as set out above according to the sample type and the individual lysis solution.

The liver sample can be replaced with other tissue and cell types as set out by the manufacturer kit instructions such as liver, spleen, brain, muscle, heart, oesophagus, testis, ovaries, thymus, kidneys, skin, intestine, pancreas, adrenal glands, lungs, bone marrow or cells such as COS-7, NIH/3T3, HeLa, 293, and CHO cells or even liquid samples such as serum, plasma or blood.

TABLE 2
Improved stability of β-actin mRNA as determined by
Q-RT-PCR following storage of a lysed rat liver
sample for 1 or 8 days at 37° C.

| Conditions | Time | | % Q-RT-PCR |
|---|---|---|---|
| RLT | 0 | | 100 |
| RLT | 1 | day | 38.4 |
| RLT | 8 | days | 4.2 |
| RLT + 8.2 mM CuCl₂ | 0 | | 100 |
| RLT + 8.2 mM CuCl₂ | 1 | day | 48.6 |
| RLT + 8.2 mM CuCl₂ | 8 | days | 22.3 |

### RNA Stabilisation in Serum and Plasma Lysates

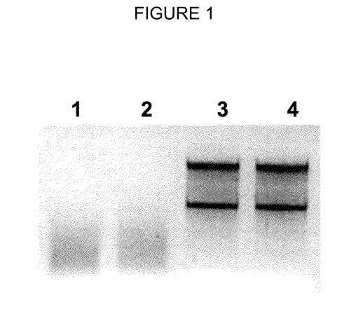

The amount of free circulating RNA in serum and plasma is too small to easily quantify, therefore in this example, an exogenous external total RNA control was added as a indicator of both RNA yield and integrity. To 600 μl of Buffer RLT (QIAGEN) was added 10 μl of a 0.5M solution of a metal or metal salt such as CuCl₂ (Sigma-Aldrich Cat. No. 203149) to give a final concentration of approximately 8 mM CuCl₂ and briefly mixed. To the guanidine/metal salt mixture was added 60 μl of freshly thawed human serum or plasma (Sigma-Aldrich) and mixed by pipetting gently several times before adding 5 μl (1.2 μg/μl) of rat total control RNA, mixing and incubating for 22 hours at 37° C. The RNA was then purified according to manufacturer's instructions (QIAGEN RNeasy Mini Kit, Cat. No. 74106) and elution in 100 μl of water. The yield and purity of the RNA was then compared by OD 260/280 nm and the integrity of the RNA determined by agarose gel analysis. These results are shown in Table 3 and FIG. 1.

TABLE 3
RNA yield and purity from plasma and serum

| | | | Total Yield | |
|---|---|---|---|---|
| | | Time | μg | OD |
| Lane | Conditions | (hours) | (% Yield) | 260/280 |
| 1 | RLT + serum | 22 | 1.47 (25) | 2.13 |
| 2 | RLT + plasma | 22 | 1.17 (20) | 1.93 |
| 3 | RLT + serum + 8.2 mM CuCl₂ | 22 | 4.66 (78) | 2.15 |
| 4 | RLT + plasma + 8.2 mM ₂ | 22 | 4.45 (74) | 2.17 |

### RNA Stabilisation in Plant Tissue Lysates

To 600 μl of Buffer RLT (QIAGEN) containing 6 μl 14.3M β-mercaptoethanol, was added 10 μl of a 0.5M solution of a metal or metal salt such as CuCl₂ (Sigma-Aldrich Cat. No. 203149) to give a final concentration of approximately 8 mM and briefly mixed. To the guanidine/mercaptoethanol/metal salt mixture was added 2-30 mg of plant tissue such as palm leaf or root, tobacco leaf or maize root and the tissue homogenised according to manufacturer's instructions (QIAGEN RNeasy Mini Kit, Cat. No. 74106). 600 μl portions of the lysate were then purified immediately according to manufacturer's instructions or stored for 1 or 8 days at 37° C. before purification and elution in 100 μl of water. The yield and purity of the RNA was then compared by OD 260/280 nm and the integrity of the RNA determined by agarose gel analysis. A significant improvement in RNA integrity was obtained using CuCl₂.

### RNA Stabilisation in Bacterial Lysates

To 600 μl of Buffer RLT (QIAGEN) containing 6 μl 14.3M β-mercaptoethanol, was added 10 μl of a 0.5M solution of a metal or metal salt such as CuCl₂ (Sigma-Aldrich Cat. No. 203149) to give a final concentration of approximately 8 mM CuCl₂ and briefly mixed. A pellet derived from 200 μl of a liquid over-night culture of E. coli was lysozyme treated and then mixed in the guanidine/mercaptoethanol/metal salt mixture. 600 μl portions of the lysate were then purified immediately according according to manufacturer's instructions (QIAGEN RNeasy Protect Bacteria Mini Kit, Cat. No. 74524). or stored for 60 hours at 37° C. before purification and elution in 100 μl of water. The yield and purity of the RNA was then compared by OD 260/280 nm and the integrity of the RNA determined by agarose gel analysis. The bacterial samples stored with the addition of a final concentration 8.2 mM CuCl₂ or FeCl₃ were markedly less degraded than without addition.

### RNA Stabilisation in Yeast Lysates

To 600 μl of Buffer RLT (QIAGEN) containing 6 μl 14.3M β-mercaptoethanol, was added 10 μl of a 0.5M solution of a metal or metal salt such as CuCl₂ (Sigma-Aldrich Cat. No. 203149) or FeCl₃ (Sigma-Aldrich Cat. No. 451649) to give a final concentration of approximately 8 mM metal ion and briefly mixed. A pellet derived from 100 μl of a liquid over-night culture of S. cerevisiae was zymolase treated and then dispersed into the guanidine or the guanidine/metal salt mixture. 600 μl portions of the lysate were then purified immediately according according to manufacturer's instructions (QIAGEN RNeasy Mini Kit, Cat. No. 74106). or stored for 60 hours at 37° C. before purification and elution in 100 μl of water. The yield and purity of the RNA was then compared by OD 260/280 nm and the integrity of the RNA determined by agarose gel analysis. The yeast samples stored with the addition of a final concentration 8.2 mM CuCl₂ or FeCl₃ were markedly less degraded than without addition.

### Viral RNA Stabilisation for Molecular Testing

To test the advantage of adding a metal salt to guanidine lysis solutions for viral RNA purification, a COBAS AMPLICOR HIV-1 MONITOR™ Test, version 1.5 (Roche Diagnostics) was used. To the HIV-1 LYS solution (Roche Diagnostics) containing 68% guanidine thiocyanate was added CuCl₂ to a final concentration of 8 mM before use. The manufacturer's instructions were then followed, briefly, 100 μl of HIV-1 QS was added to each tube of HIV-1 LYS and mixed, 600 μl of the active HIV-1 Lysis solution was added to a fresh tube and 200 μl of HIV-1 positive clinical sample (approximately 10^{e}5 virus particles per ml) was added, vortexed 3-5 seconds and then incubated 10 minutes at ambient temperature. After the addition of 800 μl of isopropanol, the manufacturer's instructions were again followed until the end of the test. An additional test was run by increasing the incubation time from 10 minutes at ambient to 6 hours at 37° C. before proceeding with the remainder of the protocol according to manufacturer's instructions. Whilst the addition of the metal salt did not have a significant affect on the detection sensitivity of the test, it was found that following incubation of the HIV-1 Lysis solution with the HIV-1 positive clinical sample, a significant increase in detection sensitivity was obtained. Note: if the blood draw tube contained EDTA it may be necessary to calibrate the amount of metal salt such as CuCl₂ necessary to saturate the EDTA chelator and obtain a final concentration of approximately 6-8 mM.

RNA Purification from Animal Tissues Using Arginine as the Chaotropic Lysis Solution

We tested whether Arginine could serve as a ‘green’ non-toxic chaotropic alternative to guanidine. Instead of using RLT lysis buffer (QIAGEN), 30 mg of animal tissue such as rat liver was added to 600 μl of 2.7M L-Arginine pH 7.0 (Sigma-Aldrich Cat. No. 11009) and homogenised according to the manufacturer's instructions (QIAGEN RNeasy Mini Kit, Cat. No. 74106). The Arginine lysate tended to form two phases, the yield of the Arginine purified sample was less than with RLT but sufficient to demonstrate that it could serve as a chaotropic alternative to guanidine.

Increased RNA Degradation in Guanidine Tissue Lysates with Chelators

To 600 μl of Buffer RLT (QIAGEN) was added concentrated EDTA to give a final concentration of 8 mM or 200 mM and then briefly mixed. To the guanidine/EDTA mixture was added 4-30 mg of rat liver and the tissue homogenised according to manufacturer's instructions (QIAGEN RNeasy Mini Kit, Cat. No. 74106). 600 μl portions of the lysate were then stored for 1 or 8 days at 37° C. before purification and elution in 100 μl of water. The yield and purity of the RNA was then compared by OD 260/280 nm and the integrity of the RNA determined by EtBr agarose gel electrophoresis. It was found that the addition of either 8 mM or 200 mM EDTA significantly increased the rate of RNA degradation compared with guanidine alone controls. Therefore, contrary to what is commonly assumed to be an RNA protective activity of EDTA, it has a deleterious effect on RNA quality.

### RNA Stabilisation in Animal Tissue Lysates Using a Promega SV™ Total RNA Isolation System

To 2 ml of Solution RLA ‘RNA Lysis Solution’ (Promega Cat. No. Z3101) was added β-mercaptoethanol to a final concentration of 1%. A 400 mg piece of rat liver was frozen in liquid nitrogen, ground with a mortar and pestle and 100 mg of the frozen powder added to each of four tubes containing 175 μl of the RLA/β-mercaptoethanol solution and mixed by inversion, then to each tube 350 μl of RNA Dilution Buffer (Promega Cat. No. Z3101) was added and mixed by inversion. Added to two of the four lysate tubes was a 50× solution of CuCl₂ to give a final concentration of 8.2 mM CuCl₂.

All four tubes were heated for 3 minutes at 70° C. to ‘improve yield’ according to the manufacturer's instructions, then two tubes, +/−CuCl₂ were immediately purified, and the two other tubes +/−CuCl₂ were incubated for 2 days at 37° C. and then purified according to the kit instructions (Promega SV Total RNA Isolation System, Cat. No. Z3101) and eluted in 100 μl of water. Following EtBr 1% agarose gel electrophoresis of all four samples, it was found that the RNA purified immediately from the rat liver lysates treated with CuCl₂ were less degraded than the lysates containing no CuCl₂. It is important to note that the manufacturer's protocol requires heating the RNA containing guanidine lysate at 70° C. for 3 minutes which consequently negatively impacts the quality of the RNA, indeed it is stated in the protocol that “Incubating longer than 3 minutes may compromise the integrity of the RNA”. It has been found that the addition of CuCl₂ minimises degradation during this step and it has been found that, in the presence of 8 mM CuCl₂ it is even possible to increase this incubation step to 5 minutes or more at 70° C. thereby increasing RNA yields from difficult to lyse tissues such as heart and skeletal muscle. Furthermore, it was also found that using CuCl₂ protects RNA from degradation during incubation for 2 days at 37° C. Therefore, surprisingly it is possible to not only improve the storage properties of guanidine lysis solutions but also to improve the quality of RNA purified according to the manufacturer's instructions.

### RNA Stabilisation in Animal Tissue Lysates Using the GE Healthcare Illustra™ RNAspin Mini Kit

To 2 ml of Solution RA1 (GE Healthcare Cat. No. 25-0500-70) was added β-mercaptoethanol to a final concentration of 1%, to 1 ml of this solution was added a 50× solution of CuCl₂ to give a final concentration of 8.2 mM CuCl₂, the other 1 ml had nothing more added. A 100 mg piece of rat liver was homogenised in each of the two 1 ml tubes using a Polytron. Two tubes each containing 350 μl of the RA1/β-mercaptoethanol/rat liver lysate and two tubes with the added CuCl₂ were prepared, and then two of these tubes (+/−CuCl₂) were immediately purified and the other two (+/−CuCl₂) incubated for 18 hours at 37° C. and then purified according to the kit instructions GE Healthcare Illustra™ RNAspin Mini Kit Cat. No. 25-0500-70) and eluted in 100 μl of water. Following EtBr 1% agarose gel electrophoresis of all four samples, it was found that the RNA from the rat liver lysates treated and incubated with CuCl₂ were far less degraded than the lysates containing no CuCl₂ demonstrating that this invention can also be used to protect RNA in a variety of commercialised RNA extraction kits.

### RNA Stabilisation During Purification Using GE Healthcare Illustra™ RNAspin Mini Kit

To 2 ml of Solution RA1 (GE Healthcare Cat. No. 25-0500-70) was added β-mercaptoethanol to a final concentration of 1%, a 100 mg piece of rat liver was added and homogenised using a Polytron. Two tubes each containing 350 μl of the RA1/β-mercaptoethanol/rat liver lysate were immediately purified according to the kit instructions except that 8 mM final concentration of CuCl₂ was added to wash solutions RA2 and MDB both of which contain guanidine thiocyanate (GE Healthcare Illustra™ RNAspin Mini Kit Cat. No. 25-0500-70) and eluted in 100 μl of water. Following EtBr 1% agarose gel electrophoresis of all four samples, it was found that the RNA from the rat liver lysates purified using CuCl₂ in the wash solutions RA2 and MDB were slightly superior in quality to the regular wash solution demonstrating that metal salts can also be used to protect RNA during purification using commercialised RNA extraction kit wash solutions.

### RNA Stabilisation in Animal Tissue Lysates Using Invitrogen Purelink™ Micro-to-Midi Total RNA Kit

To 2 ml of ‘RNA Lysis Solution’ (Invitrogen, Cat. No. 12183-018) was added β-mercaptoethanol to a final concentration of 1% and then 100 mg of fresh-frozen rat liver was briefly homogenised using a Polytron (Kinematica) or a Dounce homogeniser. Two portions of 600 μl were immediately removed and to one lysate tube, CuCl₂ was added to a final concentration of 8.2 mM. Both tubes were incubated for 2 days at 37° C. and then purified according to the kit instructions (PureLink™ Micro-to-Midi Total RNA Kit, Invitrogen, Cat. No. 12183-018) and eluted in 1000 of water. Following EtBr 1% agarose gel electrophoresis, it was found that the RNA from the rat liver lysate treated with CuCl₂ was significantly less degraded than the lysate containing no CuCl₂, therefore the stabilising ability on RNA of CuCl₂ in guanidine lysates is general to several commercialised kits and reagents. Similar results were obtained by adding 8 mM final CuCl₂ concentration to buffer L3 (Lysis Buffer) from the kit PureLink™ FFPE Total RNA Isolation Kit for rapid purification of total RNA from formalin-fixed, paraffin-embedded (FFPE) tissues (Invitrogen Cat. No. K1560-02) and storing the FFPE rat liver sample lysate at 37° C. for 18 hours.

### FFPE Extraction Using Guanidine-Metal Mixtures

Formalin fixed tissues such as FFPE cancer biopsies are a rich source of RNA for diagnostic tests, unfortunately formalin leads to the cross linking of the RNA nucleobases rendering the modified RNA difficult or even impossible to detect by hybridisation or amplification (e.g. Q-RT-PCR). Therefore it is necessary to reverse the cross-links on the RNA prior to analysis but to do so requires elevated temperatures or harsh chemicals. Generally the elevated temperatures that are necessary such as 15 minutes at 50° C. and then 15 minutes at 80° C. (RecoverAll™ Total Nucleic Acid Isolation Kit, Ambion) or 10 minutes at 72° C. and then 60° C. for 10-60 minutes or “extend the incubation time by an additional 30-60 minutes and up to 3 hours, until lysis is complete” (PureLink™ FFPE Total RNA Isolation Kit, Invitrogen) result in compromised mRNA quality, indeed the manufacturer's make cautionary remarks such as “Extending the incubation at 80° C. substantially (more than 2 min) may result in RNA degradation” and “Because the RNA extracted from fixed tissues is likely to be degraded, plan to analyze small amplicons”, or “Place in a water bath or heating block at 70° C. for 3 minutes. Incubating longer than 3 minutes may compromise the integrity of the RNA”.

In order to avoid RNA degradation during reversal of the cross links, the tissue sample can be heated at 50, 60, 70 or even 80° C. for 5-60 minutes in a mixture of guanidine/metal salt such as 5M guanidine thiocyanate/8 mM CuCl₂ prior to RNA purification using standard methods such as RNeasy Micro kit (QIAGEN).

### Storage and Transport of RNA in the Absence of a Cell or Tissue Lysate

Even in the absence of RNases, pure RNA in pure guanidine is significantly more stable than pure RNA in water, the addition of a metal salt such as final concentration 8 mM CuCl₂, to the guanidine can stabilise the RNA sample even more than in guanidine alone. The RNA in the guanidine/metal salt mixture can then be stored and transported at room temperature or on ice rather than frozen on dry ice. This is useful for example for the transport and storage of molecular weight standards or internal controls such as the HIV-1 internal control (IC) that is part of the AMPLICOR® HIV-1 Test (Roche Molecular Diagnostics).

To 1 ml of 5M guanidine thiocyanate was added CuCl₂ to a final concentration of 8 mM and mixed. Portions of 100 μl of this mixture were then added to 10 ng to 10 μg of the RNA sample to be stabilised and mixed. The RNA solution can then be stored and transported at room temperature and portions of the RNA can be conveniently added directly to the clinical sample prior to purification of the clinical RNA such as HIV containing blood or plasma. Advantageously, unlike when using guanidine alone, the RNA does not need to be particularly pure and free of amines, therefore crude RNA preparations can be used such as those having OD 260/280 ratios of less than 1.8, or even those less than 1.6 or 1.4. Conveniently RNA bacteriophages useful for assay internal controls that are also composed of protein and RNA such as MS2 and in Armored RNA® (Ambion) can be added directly to the guanidine/metal salt mixture without further purification of the RNA. In the case of the molecular weight standard, the guanidine can be diluted before loading in the agarose or acrylamide gel and electrophoresis.

### Protection of RNA in Tissue Samples During Lysis

The lysis of tissue samples is a rate limiting step of most RNA purification protocols. Tissue samples such as muscle, heart and skin that are rich in structural proteins such as collagen, actin, myosin, keratin, elastin or other samples such as RNAlater™ preserved tissues which are more difficult to homogenise compared with fresh tissues, bone, soil, yeast or bacteria can require extreme means to disrupt even using guanidine lysis buffers. Relatively easy to lyse samples such as fresh liver and brain, if high throughput is required are often lysed using semi-automated mechanical means. Commonly, mechanical means such as a TissueRuptor® or TissueLyser II® (QIAGEN), OMNI Ruptor® (OMNI International, a Precellys®24 (BERTIN Technologies), Polytron (Brinkmann), Tekmar Tissuemizer® (Tekmar Tissuemizer Co), Omni-Mixer® (SORVALL) are used. Unfortunately, such mechanical methods, especially when ceramic or steel beads are used to facilitate disruption lead to friction and significant heating of the sample. Heating the lysate in the presence of guanidine leads to rapid degradation of the RNA. In order to reduce such unwanted RNA degradation, the extent and intensity of the mechanical disruption must be limited and even cooling systems in the equipment must be used. However, it has been found that by using metals and metal salts in the guanidine that the mechanical disruption can be more intense allowing shorter run times and higher sample throughput.

To 600 μl of Buffer RLT containing 6 μl 14.3M β-mercaptoethanol, was added 10 μl of a 0.5M solution of a metal or metal salt such as CuCl₂ to give a final concentration of approximately 8 mM CuCl₂. To the guanidine/mercaptoethanol/metal salt mixture was added 30 mg of rat liver and an identical control but without the metal salt prepared, the tissue was homogenised mechanically using a TissueLyser II®, 4×5 minutes at 25 Hz (QIAGEN). The RNA was then purified according to manufacturer's instructions (QIAGEN RNeasy Mini Kit, Cat. No. 74106). It was found that the samples prepared using CuCl₂ in the lysis solution were significantly less degraded than those prepared using the standard guanidine. Therefore, even when the purpose of the protocol is not to store the lysate but simply to extract RNA from a tissue using mechanical means, there is a significant advantage to using metal or metal salts in the guanidine lysis solution. In this way, the intactness of the RNA is improved and throughput can be increased. It will be evident to one skilled in the art that it will be possible to combine this advantage with manual, semi-automated (e.g. Maxwell® (PROMEGA), QIAcube (QIAGEN) MagNA Pure Compact RNA Isolation with MagNA Pure Compact Instrument (Roche Diagnostics GmbH, Germany), or fully automated RNA purification systems (NucliSens® easyMAG™ bio-robot (Biomérieux).

Protection of HIV RNA in bDNA Assays

The entire HIV-1 bDNA assay was performed according to the manufacturer's instructions except CuCl₂ was added to one set of Lysis working reagent (Bayer VERSANT bDNA 3.0 assay) to a final concentration of 8.2 mM. Lysis working reagent (+/−CuCl₂) was added to a HIV virus pellet, followed by vortexing for 20 s. 2 h of incubation in a 63° C. heat block, transfer of viral lysate to a 96-well capture plate, and transfer of the plate to System 340 programmed for the HIV RNA 3.0 setting.

### Ninhydrin Assay to Determine Free Amine Content of a Sample

4 μl Ninhydrin (50 mg/ml in water) was added to 400 of the guanidine RLT buffer (Qiagen) containing 1 mg homogenised rat liver and mixed. The colour change was determined after incubating for 15 minutes at 20° C. by spectrometry at 570 nm. Identical mixtures can be prepared and tested by adding variable amounts and types of metal ions and salts, the reduction in absorbance at 570 nm indicates that amines are being complexed or chelated by the metal ions thereby providing a simple screen to determine potentially useful metal ions and salts and their appropriate concentrations of use. Appropriate final concentrations of metal ions or salts can be tested in the range of 0.1-20 mM and more preferably less than 10 mM. It should be noted that the Ninhydrin test can provide only approximate results and that empirical tests of RNA quality and yield are preferred to determine the optimum use of the metal ion or salt.

Alternatively the concentration of the primary or secondary amine in the biological sample can be determined by preparing a standard curve with known amounts of amine in RLT and comparing with the unknown sample which may help to optimise adding the correct amount of metal ion or salt to samples containing particularly high concentrations of amine.

### Demonstration of RNA Degradation Using Pure Amine in Guanidine

To a pre-purified source of RNA such as 2 μg of total rat liver RNA was added 50 μl 6M solution of guanidine HCl or buffer RLT (Qiagen), varying final concentrations of amines such as 30 μM ethylenediamine, 300 μM Lysine, 300 μM Histidine, 300 μM glycine or 20 μg of a protein such as BSA. The mixture was then heated at 60-70° C. for 30-90 minutes before purifying the RNA with a silica spin-column (QIAprep, Qiagen) and assessing the extent of degradation by agarose gel electrophoresis. Conveniently, the protective effects of adding metal ions such as CuCl₂ can also be determined in the same manner by making the same mixture of RNA/guanidine/amine, then adding a source of metal ions such as CuCl₂ prior to the heating step, purification and RNA analysis. In this manner it is possible to screen for the most appropriate metal ions that provide RNA stability in a guanidine/amine mixture.

### Empirical Identification of Appropriate Metal Ions and Salts

Whilst synthetic mixtures of guanidine/amine and RNA are a convenient and well defined test for RNA stability with metal ions and salts, a more appropriate method is to use tissue and cell lysates or blood derivatives such as serum. 200 mg of rat liver was homogenised using a Polytron in 6 ml of guanidine RLT buffer (Qiagen) and 600 μl portions of the lysate were added to individual 1.5 ml tubes. To each tube was added a variable amount or type of metal ion or salt such as AgCl, AgCO₂CH₃, CuCl, CuCl₂, CuCO₂CH₃, Cu(CO₂CH₃)₂, FeCl₂, FeCl₃, InCl, InCl₂, InCl₃, In(CF₃SO₃)₃, ErCl₃, Er₂(C₂O₄)₃, Er(CF₃SO₃)₃, ZnSO₄, ZnCl₂, ZnI₂, Zn₃(PO₄)₂, Zn(CO₂CH₃)₂, ZrCl₄, ZrF₄ or a mixture such as CuCl₂/FeCl₂ to give a final metal ion concentration of 0.1-20 mM but more preferably 2-12 mM.

The mixture was then incubated at 60-70° C. for 30-90 minutes or at 37-42° C. for 2-21 days or 4° C. for 1-4 months before purifying the RNA with, conveniently an RNeasy Mini kit or a RNeasy-96 (Qiagen) and assessing the extent of degradation by agarose gel electrophoresis or Bioanalyser 2100 (Agilent).

It will be apparent to one skilled in the art that the type and concentration of the metal ion or metal salt is not particularly limited other than excluding Group 1 and 2 elements and preferably has the following attributes, is: soluble, stable in guanidine, does not precipitate with beta-mercaptoethanol, non-toxic, not expensive, reduces RNA degradation in many or even all types of biological samples regardless of their source, transparent or lightly coloured in guanidine, does not negatively affect RNA binding to silica surfaces, reduces or does not affect contaminant binding, does not degrade RNA by catalysis or depurination, trace amounts do not inhibit molecular assays and enzymes such as reverse transcriptase and allows the parallel purification of DNA and/or proteins if desired. It will also be apparent that in order to determine the optimum selection and concentration of metal ion or salt, empirical tests such as those are set out in this example will have to be carried out with a variety of sample types such as blood, tissue and cells.

While certain embodiments have been described in terms of the preferred embodiments, it is understood that variations and modifications will occur to those skilled in the art. Therefore, it is intended that the appended claims cover all such equivalent variations that come within the scope of the following claims.

1. A method for stabilising RNA in an RNA-containing sample, which method comprises contacting the sample with guanidine and a metal ion to form a stabilised RNA-containing composition in which the metal ion is present at a concentration which is no more than 20 mM, wherein the metal ion is derived from a metal other than from a Group 1 or Group 2 metal.

2. A method according to claim 1, wherein the metal ion is an ion of copper, zinc, iron, zirconium, erbium, indium, terbium, silver, gold, aluminium, tin, bismuth, lead or vanadium.

3. A method according to claim 2, wherein the metal ion is provided as a salt selected from CuCl₂, Cu(CO₂CH₃)₂, CuCl, AuCl, FeCl₃, ZrCl₄, TbCl₃ and (CF₃SO₃)₃In.

4. A method according to any one of claims 1, wherein the metal ion concentration in the stabilised RNA-containing composition is less than 10 mM.

5. A method according to claim 1, wherein the metal ion concentration in the stabilised RNA-containing composition is at least 2.5 mM.

6. A method according to claim 1, wherein the guanidine concentration in the stabilised RNA-containing composition is at least 2M and no more than 8M.

7. A method according to claim 1, wherein the RNA-containing sample comprises blood, serum or plasma.

8. A method according to any one of claim 1, wherein the RNA-containing sample comprises a tissue selected from liver, spleen, brain, muscle, heart, oesophagus, testis, ovaries, thymus, kidneys, skin, intestine, pancreas, adrenal glands, lungs, bone marrow, or a cancer sample, tumour or biopsy, or a plant tissue selected from leaves, flowers, pollen, seeds, stems and roots of rice, maize, sorghum, palm, vines, tomato, wheat, barley, tobacco, sugar cane and Arabidopsis, or a bacteria selected from E. coli, Staphylococcus, Streptococcus, Mycobacterium, Pseudomonas, and bacteria that cause Shigella, Diptheria, Tetanus, Syphilis, Chlamydia, Legionella, Listeria and leprosy.

9. A method according to claim 1, wherein the RNA is viral RNA, mRNA or miRNA.

10. A method according to claim 9, wherein the RNA-containing sample includes an animal RNA virus selected from Norwalk, Rotavirus, Poliovirus, Ebola virus, Marburg virus, Lassa virus, HIV, HCV, Hantavirus, Rabies, Influenza, Yellow fever virus, Corona Virus, SARS, West Nile virus, Hepatitis A, C(HCV) and E virus, Dengue fever virus, toga, Rhabdo, Picorna, Myxo, retro, bunya, corona and reoviruses.

11. A method according to claim 1, which includes a step of lysing the RNA-containing sample in the presence of the guanidine and the metal ion.

12. A method according to claim 11, wherein the stabilised RNA-containing composition is used in a bDNA assay.

13. A method according to claim 1, wherein the stabilised RNA-containing composition is contacted with a solid phase binding surface for binding the RNA.

14. A method according to claim 13, wherein the solid phase binding surface comprises silica.

15. A composition for extracting RNA from a biological sample, which composition comprises guanidine and a source of metal ions for mixing with the sample to provide a metal ion concentration of no more than 20 mM whereby the RNA is stabilised against degradation, wherein the metal ion is derived from a metal other than from a Group 1 or Group 2 metal.

16.-59. (canceled)

60. A stabilised RNA-containing composition, which comprises RNA, guanidine and a metal ion, wherein the metal ion is present at a concentration which is no more than 20 mM and the metal ion is derived from a metal other than from a Group 1 or Group 2 metal.

61.-68. (canceled)

69. A method for stabilising RNA in an RNA-containing sample using a chaotrope and a metal ion at a final concentration 0.1 mM to 20 mM in the sample.

70.-84. (canceled)

85. A composition for extracting RNA from a biological sample, the composition comprising a chaotrope and a source of metal ions for mixing with the sample to produce a mixture, wherein the metal ions are present in the mixture at a concentration of between about 0.1 mM to about 20 mM.

86.-108. (canceled)

109. A stabilised RNA-containing composition comprising RNA, guanidine, and a metal ion at a concentration of about 0.1 mM to about 20 mM.

110.-127. (canceled)

128. A method for maintaining the integrity of RNA within a biological sample, the method comprising adding to the sample at least type of one metal ion to a final concentration of about 0.1 mM to about 20 mM and a chaotrope.

129.-145. (canceled)
